SEQUENCE LISTING (13 sequences)

SEQ ID NO: 1 - protein, 194 aa, Homo sapiens
  MISC_FEATURE (1)..(194): PGRMC1
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 2 - DNA, 6047 bp, Homo sapiens
  misc_feature (1)..(6047): coding strand of expression vector pcDNA3_ PGRMC1_3HA
aaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattctagatacccatacgatgttcctgactatgcgggctatccctatgacgtcccggactatgcaggatcctatccatatgacgttccagattacgcttctagatgaactagagggcccgttt

SEQ ID NO: 3 - protein, 229 aa, Homo sapiens
  MISC_FEATURE (1)..(229): PGRMC1_3HA
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 4 - protein, 229 aa, Homo sapiens; identical to SEQ ID NO: 3
  MISC_FEATURE (1)..(229): PGRMC1_3HA

SEQ ID NO: 5 - protein, 229 aa, Homo sapiens
  MISC_FEATURE (1)..(229): PGRMC1_3HA_S56A
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 6 - protein, 229 aa, Homo sapiens
  MISC_FEATURE (1)..(229): PGRMC1_3HA_S180A
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 7 - protein, 229 aa, Homo sapiens
  MISC_FEATURE (1)..(229): PGRMC1_3HA_S56A/S180A
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 8 - protein, 229 aa, Homo sapiens
  MISC_FEATURE (1)..(229): PGRMC1_3HA_S56A/C128S/S180A
AAEDVVATGADPSDLESGGLLHEIFTSPLNLLLLGLCIFLLYKIVRGDQPAASGDADDDEPPPLPRLKRRDFTPAELRRFDGVQDPRILMAINGKVFDVTKGRKFYGPEGPYGVFAGRDASRGLATFSLDKEALKDEYDDLSDLTAAQQETLSDWESQFTFKYHHVGKLLKEGEEPTVYADEEEPKDESARKNDSRYPYDVPDYAGYPYDVPDYAGSYPPYDVPDYASR

SEQ ID NO: 9 - protein, 229 aa, Homo sapiens
  MISC_FEATURE (1)..(229): PGRMC1_3HA_Y138F
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEFDDLSDLTAAQQETLSDWESQFTFKYHHVGKLLKEGEEPTVYSDEEEPKDESARKNDSRYPYDVPDYAGYPYDVPDYAGSYPPYDVPDYASR

SEQ ID NO: 10 - protein, 229 aa, Homo sapiens
  MISC_FEATURE (1)..(229): PGRMC1_3HA_Y179F
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVFSDEEEPKDESARKNDSRYPYDVPDYAGYPYDVPDYAGSYPPYDVPDYASR

SEQ ID NO: 11 - protein, 229 aa, Homo sapiens
  MISC_FEATURE (1)..(229): PGRMC1_3HA_Y179F/S180A
AAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVFADEEEPKDESARKNDSRYPYDVPDYAGYPYDVPDYAGSYPPYDVPDYASR

SEQ ID NO: 12 - DNA, 6047 bp, Homo sapiens
  misc_feature (1)..(6047): complimentary strand of expression vector pcDNA3_PGRMC1_3HA
aaacgggccctctagttcatctagaagcgtaatctggaacgtcatatggataggatcctgcatagtccgggacgtcatagggatagcccgcatagtcaggaacatcgtatgggtatctagaatcatttttccgggcactctcatcttttggttcttcctcatctgagtacacagtgggctcctccccctccttcagcagtttgcccacgtgatgatacttgaaagtgaactgagactcccagtcactcagagtctcctgctgggcagcagtgaggtcagaaaggtcatcgtactcatccttcagtgcttccttatccaggcaaaatgtggcaaggcccctggatgcatctcttccagcaaagaccccatacggcccctcgggcccgtagaatttgcggcctttggtcacatcgaacaccttgccgttgatggccatgagtatgcgcgggtcctggacgccgtcgaagcgccgcagctcggcgggggtgaagtcgcgccgcttgaggcggggcagagggggcggctcgtcgtcgtcgctgtcgccgctggccgccggctggtccccgcgcacgatcttgtagagcaggaagatgcagaggccaagcagcagcaggttgagcggcgacgtgaaaatctcatgcagcagcccgccgctctccagatcgcttgggtcggcgccagtcgccaccacatcctcggcagccatggcaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggttt

SEQ ID NO: 13 - protein, 5 aa, Homo sapiens
  MISC_FEATURE (1)..(5): Residues corresponding to untranslated codons of expression vector pcDNA3_PGRMC1_3HA
TRGPV

(19) United States
(12) Patent Application Publication
(10) Pub. No.: US 2009/0318354 A1
(43) Pub. Date: Dec. 24, 2009

(54) Use of at least one isoform of progesterone receptor membrane component 1 (PGRMC1)

(75) Inventors: Michael Cahill, New South Wales (AU); Hans Neuhauer, Tuebingen (DE)

Correspondence Address: ROTHWELL, FIGG, ERNST & MANBECK, P.C., 1425 K STREET, N.W., SUITE 800, WASHINGTON, DC, 20005, US

(73) Assignee: PROTEOSYS AG, Mainz (DE)

(21) Appl. No.: 12/443,092

(22) PCT Filed: Sep. 26, 2007
(86) PCT No.: PCT/EP2007/008370

§ 371 Date: Apr. 27, 2009

(30) Foreign Application Priority Data
Sep. 26, 2006 (EP) PCT/EP2006/009351
Mar. 28, 2007 (EP) 07006379.7

(51) Int. Cl. A61K 38/17; C07K 14/705; C07K 16/00; C12Q 1/00

(52) U.S. Cl. 514/12; 530/350; 530/387.1; 435/4

(57) ABSTRACT

The invention relates to the use of at least one isoform of progesterone receptor membrane component 1 (PGRMC1) as diagnostic marker and/or therapeutic target for diseases associated with neogenin and/or DCC and/or for the manufacture of a medicament for treatment of diseases associated with neogenin and/or DCC, the use of at least one reagent that influences the function of at least one isoform of PGRMC1 for the manufacture of a medicament for treatment of diseases associated with neogenin and/or DCC, the use of at least one isoform of PGRMC1 for influencing abundance or subcellular localisation of at least one class of molecules interacting with PGRMC1, an assay kit for diagnosis and/or therapy of diseases associated with neogenin and/or DCC, comprising at least one isoform of PGRMC1 and/or at least one reagent that influences the function of at least one isoform of PGRMC1, the use of at least one isoform of PGRMC1 as diagnostic marker in diagnosis for diseases associated with aberrant bio-logical phenotypes, wherein the phosphorylation status of the at least one isoform of PGRMC1 is determined/and or estimated and the use of at least one reagent for influencing the abundance and/or activity of isoforms of proteins for diagnosis and/or therapy of diseases associated with aberrant biological phenotypes, wherein said proteins are involved in protein interaction or multiprotein complexes with either at least one isoform of phosphorylated or non-phosphorylated PGRMC1.

The invention relates to the use of at least one isoform of progesterone receptor membrane component 1 (PGRMC1) as diagnostic marker and/or therapeutic target, the use of at least one isoform of progesterone receptor membrane component 1 (PGRMC1) for the manufacture of a medicament, the use of at least one reagent that influences the function of at least one isoform of progesterone receptor membrane component 1 (PGRMC1) for the manufacture of a medicament, the use of at least one isoform of progesterone receptor membrane component 1 (PGRMC1) for influencing at least one class of molecules interacting with PGRMC1, to assay kits for diagnosis and/or therapy of diseases, to the use of at least one isoform of progesterone receptor membrane component 1 (PGRMC1) as diagnostic marker in diagnosis for diseases associated with aberrant biological phenotypes and to the use of at least one reagent for influencing, in particular increasing or inhibiting, the abundance and/or activity of isoforms of proteins for diagnosis and/or therapy of diseases associated with aberrant biological phenotypes.

Breast cancer is one of the most common forms of cancer observed in women, with more than 180,000 new cases and over 40,000 deaths expected in the USA in 2007 [1]. Endogenous estrogens, which have effects on many organs, are thought to play a major role in the development of the breast, suggesting that an increased sensitivity or longer exposures to estrogens is involved in a higher risk of tumorigenesis [2-4].

The classical estrogen receptor (ERα: hereafter ER) is found in 50-80% of breast tumors and ER status is essential in making decisions about endocrine therapy with anti-estrogens which inhibit the mitogenic activity of estrogens in breast cancer. There are three classes of anti-estrogens currently in clinical use: selective estrogen receptor modulators (SERMs, e.g., tamoxifen), aromatase inhibitors and, “pure” estrogen antagonists such as fulvestrant which, like tamoxifen, binds to estrogen receptors competitively. However, in contrast to tamoxifen, fulvestrant's binding leads to rapid degradation and loss of the ER protein [5;6].

Clinically, a positive ER status correlates with favorable prognostic features, including a lower rate of cell proliferation and histological evidence of tumor differentiation. ER status is also prognostic for the site of gross metastatic spread. For reasons unknown, ER+ tumors are more likely to initially manifest clinically apparent metastases in bone, soft tissue, or the reproductive and genital tracts, whereas ER− tumors more commonly metastasize to brain and liver. Several studies have correlated ER expression to lower Matrigel invasiveness and reduced metastatic potential of breast cancer cell lines [7;8].

Moreover, when ER+ cells are implanted in nude mice, tumors appear only in the presence of estrogens and are poorly metastatic as compared to those developed from ER− breast cancer cell lines [9;10]. This paradox suggests that ER expression could be associated with or involved in pathways that hinder cancer progression.

At the mRNA level, gene expression analysis has revealed that different molecular subtypes of tumour exist within the broader groups of ER+ and ER− breast cancers, and these are associated with different clinical outcomes. ER+ tumors exist in at least two subtypes, luminal A and luminal B, which vary markedly in gene expression and prognosis [11]. Conversely, hormone-receptor-negative breast cancer comprises two distinct subtypes, the Her2 subtype and the basal-like subtype [11;12], which differ in biology and behaviour. Both of these are associated with a worse clinical outcome than ER+ tumors.

Importantly a very similar subdivision of breast cancers has been produced using immunohistochemistry to analyze patterns of protein expression in tumor sections and, which suggests that a few protein biomarkers can be used to stratify breast cancers into different groups [13;14]. One set of biomarkers is built by the family of cytokeratins (CKs). They can be grouped into the “luminal CKs” 7/8, 18, and 19 and into the “basal CKs” 5/6 and 14 [13].

In addition to these molecular portraits, it has been shown that expression patterns present in primary breast cancers are also observed in their respective metastases [15]. Other gene expression profiles have distinguished breast cancers according to the differential expression of a wound response signature. Twenty years ago, based on the observation that there are many histological similarities between the tumor microenvironment and normal wound healing, it was proposed by Dvorak that the tumor stroma is “normal wound healing gone awry” [16]. Since then it has been discovered that genes induced in a fibroblast serum-response program are expressed in tumors by the tumor cells themselves, by tumor-associated fibroblasts, or both [17]. The molecular features that define this wound-like phenotype are evident at an early clinical stage, persist during treatment, and predict increased risk of metastasis and death in breast, lung, and gastric carcinomas.

The properties and biology of the PGRMC1 protein have been recently reviewed [18]. Briefly, progesterone binding has only been observed in biological fractions containing the protein PAIRBP1 in addition to PGRMC1. Various other steroid and cholesterol-related ligands could potentially affect PGRMC1 biology. A biological role of heme binding has been implicated, and PGRMC1 has been shown to be present in protein complexes with various cytochrome P450 enzymes. Various target peptides for interaction with proteins involved with signal transduction are present in PGRMC1, and the phosphorylation of various amino acids that would regulate such interactions have been observed in living cells. PGRMC1 also contains evolutionarily conserved motifs that are present in the protein structure at positions which could indicate a function in endocytosis or other membrane trafficking events (reviewed in reference [18]).

Other homologues of PGRMC1 are known for example from Ceanorhabdidtis elegans. The Vem-1 Ceanorhabdidtis elegans homologue of PGRMC1 interacts genetically and physically with the Unc-40 transmembrane receptor that is involved in axonal guidance [19]. Unc-40 is the receptor for Unc-6, a lamin-like molecule that acts as neuronal attraction signal and is homologous to the mammalian Netrin family [25]. Unc-40 has two mammalian homologues; Deleted in Colorectal Cancer (DCC) and Neogenin. These immunoglobulin domain transmembrane proteins act as receptors for proteins of the Netrin family, which also bind receptors of the mammalian Unc-5 family, in a system of axon guidance whose underlying components are conserved between nematodes and mammals [26-30]. The repulsive guidance molecule (RGM) family are membrane-bound molecules which also signal through the Neogenin receptor [31], as well as other receptors in axon guidance [32]. Thus migrating axons are not only attracted towards sources of Netrins, but axons growing in other directions undergo programmed cell death due to the lack of Netrin. This apoptosis depends on the presence of DCC or Neogenin [33].

Furthermore, in WO2006029836 differentially abundant isoforms of PGRMC1 in breast cancers that differed in expression of the estrogen receptor (ER) were identified. It was also demonstrated that these isoforms differ in their phosphorylation status, and that the presence of certain phosphorylation sites of PGRMC1 affected cell survival in stable cloning experiments (PCT/EP2006/009351).

Surprisingly it has now been found that PGRMC1 is a central orchestrator of biological processes which determine the pathogenicity of not only cancer cells, but indeed occupies a regulatory crossroad function providing a nexus point for the regulation of higher order biological responses in multiple biological systems. As such PGRMC1 represents a suitable target molecule for pharmaceutical cancer treatment, especially of reagents which affect phosphorylation and/or intracellular localisation of PGRMC1. Furthermore, analogous applications exist in the fields of nervous system diseases, inflammatory diseases, and cardiovascular diseases.

In the scope of the present invention the following terms shall be defined:

The term “phosphorylation status” as used herein comprises the absolute or relative degree of phosphorylation of proteins and/or reagents. The term “aberrant biological phenotypes” as used herein comprises all forms of aberrant biological in vivo manifestations, for instance uncontrolled cell proliferation.

“Activity” of proteins as used herein, comprises the enzymatic activity, binding affinity and/or posttranslational activity, in particular phosphorylation.

The term “abundance” as used herein is equivalent to the expression level of proteins, in particular of mPR, being detectable with prior art methods.

The terms “neogenin” and “Deleted in colorectal cancer (DCC)” as used herein preferably shall include any family member of these receptors and/or neogenin-like and/or DCC-like receptors which can be influenced by PGRMC1.

The present invention is accordingly directed to the use of at least one isoform of progesterone receptor membrane component 1 (PGRMC1) as diagnostic marker and/or therapeutic target for diseases according to claim 1, the use of at least one isoform of progesterone receptor membrane component 1 (PGRMC1) for the manufacture of a medicament for treatment of diseases according to claim 2 and the use of at least one reagent that influences the function of at least one isoform of progesterone receptor membrane component 1 (PGRMC1) for the manufacture of a medicament for treatment of diseases according to claim 3. Preferred embodiments of use claims 1 to 3 are specified in dependent claims 4 to 13 and 19 to 21.

The present invention is further directed to the use of at least one isoform of progesterone receptor membrane component 1 (PGRMC1) for influencing abundance or subcellular localisation of at least one class of molecules interacting with PGRMC1 according to claims 14 and 15 and to assay kits according to claims 22 and 23. Preferred embodiments of use claims 14 and 15 are specified in dependent claims 16 to 18.

The present invention is further directed to the use of at least one isoform of progesterone receptor membrane component 1 (PGRMC1) as diagnostic marker in diagnosis for diseases according to claim 24 and to the use of at least one reagent for influencing, in particular increasing or inhibiting, the abundance and/or activity of isoforms of proteins for diagnosis and/or therapy of diseases according to claim 26. Preferred embodiments of use claims 24 and 26 are specified in dependent claims 25 and 27.

The wording of all claims, including dependent claims, is hereby incorporated in the description by reference.

In a particular aspect of the present invention the term “at least one isoform of progesterone receptor membrane component 1 (PGRMC1) refers to any isoform combination of PGRMC1, including all isoforms of PGRMC1.

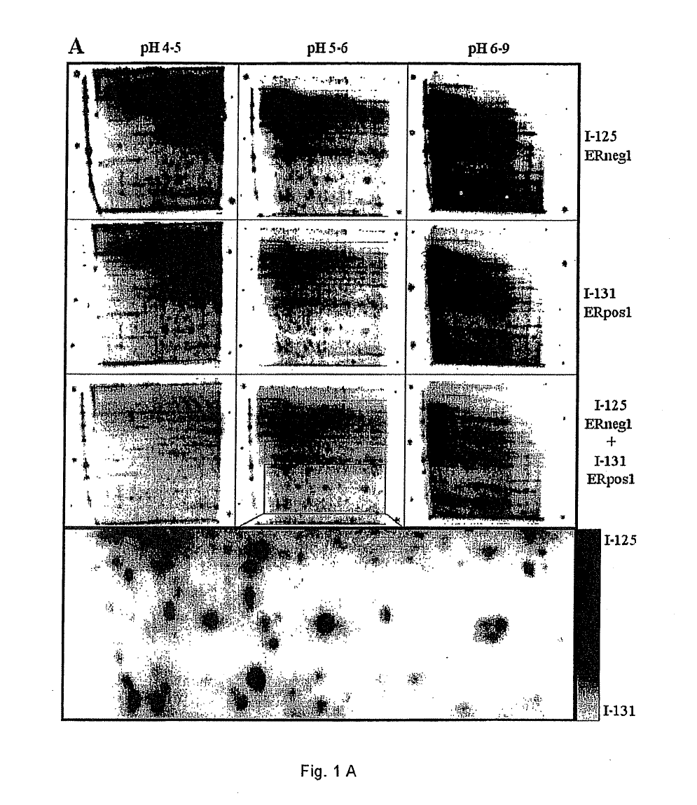

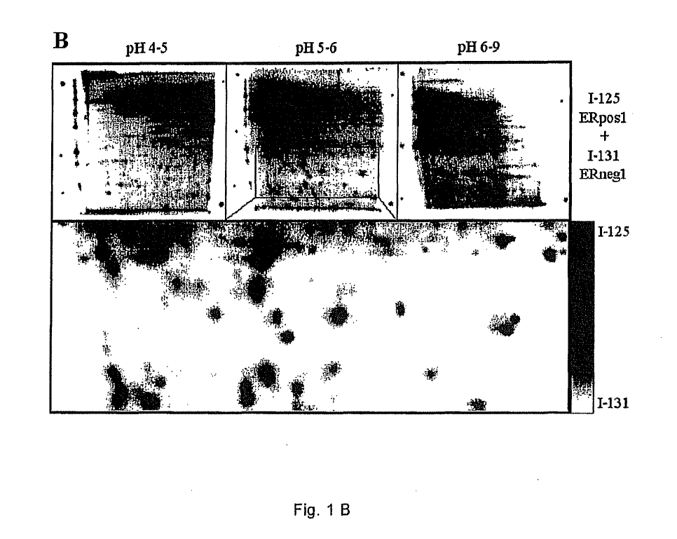

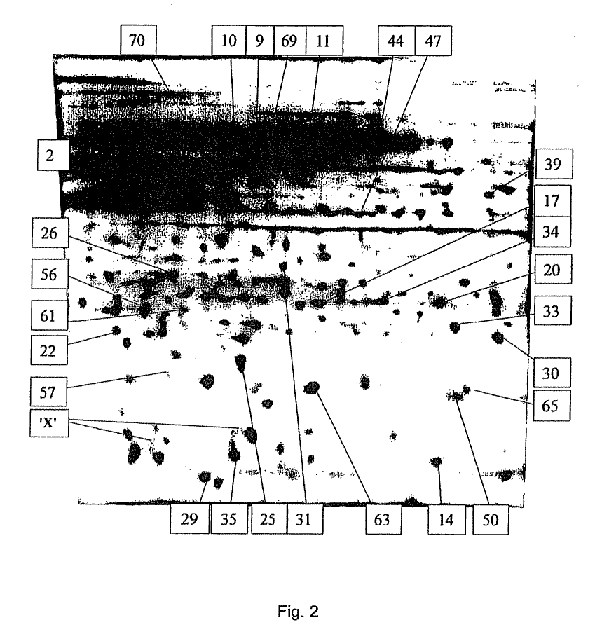

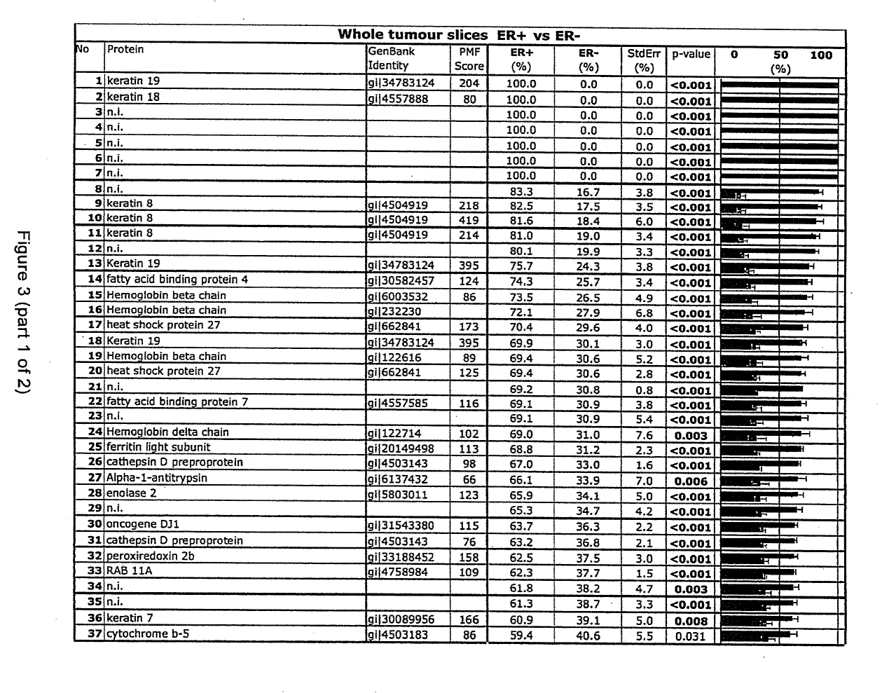

The inventors designed a paired direct comparison strategy by pooling samples derived from tissue sections from large homogenous breast tumours on the basis being either ER⁺ or ER− negative. Eight ER⁺ tumours and eight ER⁻ tumours were used. They were randomly assigned to sub-pools (table 1), each sub-pool containing normalised equal amounts of protein from two tumours. For differential analysis, sub-pool ER⁺¹ (containing T378 and T392) was differentially compared to sub-pool ER⁻¹ (containing T433 and T443), ER⁺² was compared to ER⁻², ER⁺³ was compared to ER⁻³, and ER⁺⁴ was compared to ER⁻⁴ ( an example of one inverse replicate differential analysis is presented in FIG. 1). Spots were matched across gels, and their intensities were analysed relative to ER status. Synthetic average gel images were constructed by computer (an example of which is given for the pH 5-6 experimental window in FIG. 2). The statistically most significant differential protein spots are preferably identified by mass spectrometry, in particular by MALDI-TOF (FIG. 3). In total, proteins from 325 spots were identified by MALDI-TOF PMF with MASCOT scores greater than 70, of which 72 spots represented 16 proteins that were identified in more than one protein spot (table 2). Afterwards, the proteins were identified and characterized regarding their phosphorylaton status.

Progesterone receptor membrane component 1 (PGRMC1) was identified by the inventors from three spots (table 2) that formed an approximately equidistant chain in the pH 4-5 IPG, two of which (FIG. 3: spots 52, and 62) were significantly more abundant in cancers with a negative ER status (ER⁻). Furthermore, the more acidic spots exhibited slightly retarded migration in SDS-PAGE (Sodium Dodecylsulfate Polyacrylamide Gel Elektrophoresis), consistent with possible phosphorylation differences between the spots. The putatively hypophosphorylated forms were more abundant in tumours lacking the estrogen receptor. To test the hypothesis that these quantitative differences were due to altered isoelectric points of the protein caused by differential phosphorylation in the spots, and that phosphorylation therefore may differentially affect the intracellular localisation of PGRMC1 between the test tissues, the inventors devised a phosphatase treatment regime using shrimp alkaline phosphatase (SAP). Whole cell native protein extracts from several patients were pooled and incubated with either phosphatase buffer containing SAP (+SAP), or mock incubated under identical conditions with the addition of phosphatase inhibitors but without SAP (−SAP). A raw extract that was not incubated at all prior to protein denaturation (raw) was also included in the analysis as a reference control.

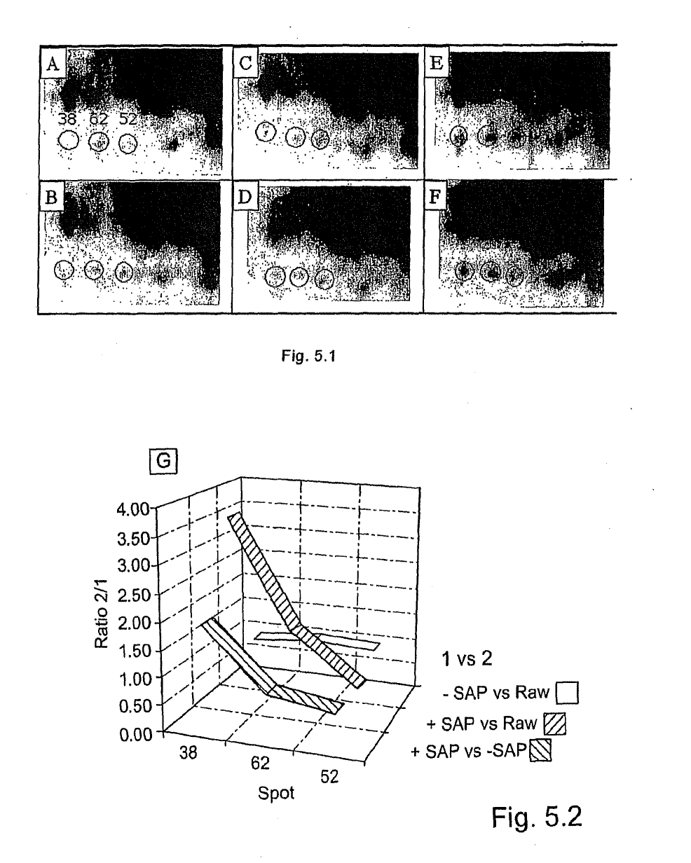

For instance, the samples were analysed by the inventors pairwise against each other by ProteoTope® imaging after inverse radioactive labelling with ¹²⁵I and ¹³¹I, and separation by daisy chain 2D-PAGE. The portions of the part of the inverse replicate gels containing the PGRMC1 spots are shown in FIG. 5. Panel A, being ¹²⁵I-labelled +SAP sample (blue) and ¹³¹I-labelled mock −SAP sample (orange) shows a discernable preponderance of ¹³¹I for the most acidic spot (spot 38). By contrast, the most basic of the spots (spot 52) exhibited a slight preponderance of ¹²⁵I. Importantly, these small differences in relative signal intensity were reproducibly detected in the inverse replicate labelled experiment of Panel B, where spot 38 also shows a discernable preponderance of ¹²⁵I and spot 52 of ¹³¹I. Panels B and C compared the phosphatase treated samples against the untreated raw extract. The same trend was observed, however the magnitude of the differences was slightly higher, the difference being possibly due to experimental error. By contrast, when the mock treatment was compared to the raw extract in panels E and F, the ratios between both samples approximated 50%. Thus, the difference in intensity of this spot was not due to the incubation, but rather due to the presence of phosphatase in the incubation. The averaged quantified results from both inverse replicate dual image gels for each sample comparison are presented graphically in Panel G. This result strongly demonstrates that the most acidic spots can be dephosphorylated, whereupon they migrate to one of the more basic spots. Taken together with the results of FIG. 3 for these three protein spots, this provided evidence that mPR is probably more highly phosphorylated in ER⁺ than ER⁻ tumours.

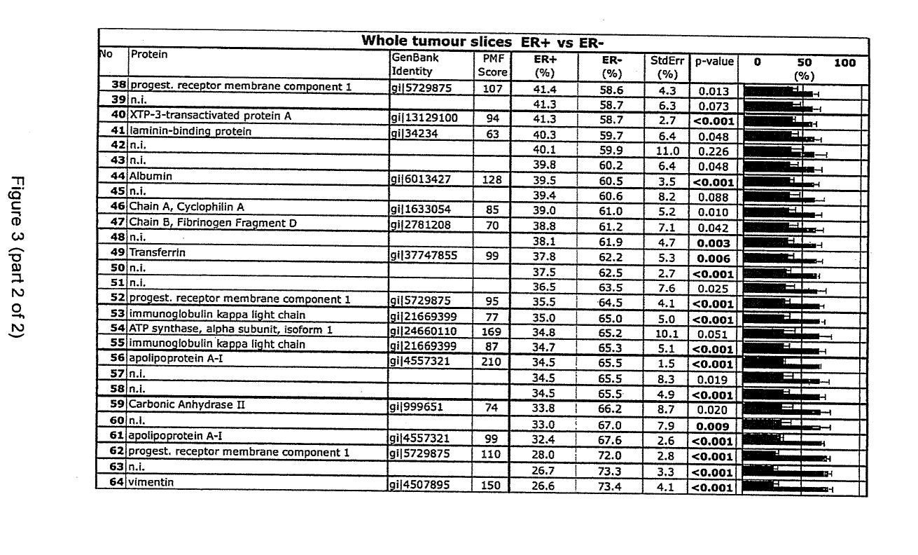

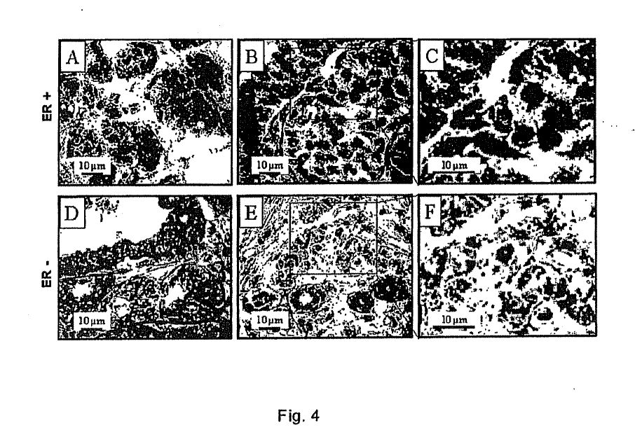

Therefore, the inventors provided evidence that the population of mPR molecules is more highly phosphorylated in ER⁺ tumours than ER⁻ tumours. Consequently, this is the first de-novo demonstration of a phosphorylation difference from primary tumours by discovery proteomics without the use of cell culture. Interestingly, this phosphorylation pattern corresponds to the presence of punctuated localised concentration of PGRMC1 in extra-nuclear regions of ER⁻ cells (FIG. 4).

According to the present invention at least one isoform of progesterone receptor membrane component 1 (PGRMC1) is used
as diagnostic marker and/or therapeutic target for diseases associated with neogenin and/or DCC and/or
for the manufacture of a medicament for treatment of diseases associated with neogenin and/or DCC.

Further, according to the present invention at least one reagent that influences the function of at least one isoform of progesterone receptor membrane component 1 (PGRMC1) is used for the manufacture of a medicament for treatment of diseases associated with neogenin and/or DCC.

According to a preferred embodiment of the invention said at least one reagent increases or decreases the function or changes the subcellular locality of PGRMC1.

According to a preferred embodiment of the invention said at least one reagent is a reagent for influencing, in particular increasing or decreasing, the phosphorylation status of the at least one isoform of PGRMC1.

According to a preferred embodiment of the invention said at least one reagent is an antibody and/or a small molecule affinity reagent directed against PGRMC1.

According to a preferred embodiment at least two or three isoforms are used as diagnostic marker and/or therapeutic target or in the manufacture of the medicament.

Further it is preferred that said diseases comprise neuronal disorders, preferably memory disorders, in particular in mammalians.

It may also be preferred that said diseases comprise cancer, in particular breast cancer, prostate cancer and/or cancer of the nervous system.

In a preferred embodiment said diseases comprise inflammatory diseases, in particular atherosclerosis or rheumatoid arthritis, and/or cardiovascular diseases.

According to a preferred embodiment of the invention at least one of said isoforms of PGRMC1 has a significantly increased abundance in cancer cells of said cancers.

According to a preferred embodiment of the invention said isoforms of PGRMC1 differ from each other in their phosphorylation status.

Further it is preferred that said cancer cells are negative for the estrogen receptor (ER−) and/or negative for the progesterone receptor (PR−).

According to the present invention at least one isoform of progesterone receptor membrane component 1 (PGRMC1) is used
for influencing, in particular increasing, abundance or subcellular localisation of at least one class of molecules interacting with PGRMC1 and/or
for influencing, in particular decreasing, abundance or subcellular localisation of at least one class of molecules interacting with PGRMC1.

According to a preferred embodiment of the invention said class of molecule is at least one of Deleted in colorectal cancer (DCC) or Neogenin.

According to a preferred embodiment of the invention said class of molecule is a ubiquitin ligase.

According to a especially preferred embodiment of the invention said class of molecule is a polyubiquitinase.

The invention is further directed to an assay kit for diagnosis and/or therapy of diseases associated with neogenin and/or DCC, comprising at least one isoform of progesterone receptor membrane component 1 (PGRMC1) and to an assay kit for diagnosis and/or therapy of diseases associated with neogenin and/or DCC, comprising at least one reagent that influences the function of at least one isoform of progesterone receptor membrane component 1 (PGRMC1).

The assay kit comprises in a further embodiment means of detection and/or discrimination of at least one isoform of PGRMC1.

Furthermore, PGRMC1 of the assay kit is phosphorylated. In particular, the assay kit may comprise at least two isoforms of PGRMC1, in particular in different phosphorylated status.

In an especially preferred embodiment the assay kit comprises plasmates encoding PGRMC1 and/or mutants of PGRMC1, especially S56A, S180A, S56A/S180A, S56A/C128S/S180A, Y138F, Y179F and/or Y179F/S180A.

According to a further aspect, an in-vivo or ex-vivo method for diagnosis of diseases related to aberrant biological phenotypes is embraced by the present invention. Said method comprises at least the step of determining the abundance and/or phosphorylation status of progesterone receptor membrane component 1 (PGRMC1) or isoforms thereof in a sample.

In a particular preferred embodiment of the present invention the isoforms of PGRMC1 are isoforms of a PGRMC1-mutant, in particular S56A, S180A, S56A, S180A, S56A, C128S, S180A, Y138F, Y179F and/or Y179F/S180A.

Normally, the samples are derived from mammals, in particular from human beings or subjects. The samples may be provided in form of biopsy or surgical extracted samples.

Preferably, the human samples are provided as microdissected human samples. The samples can be derived from a small tissue fraction, particularly from a tumor tissue fraction, especially from a breast cancer tissue fraction.

In a preferred embodiment the determined abundance and/or phosphorylation status is compared to a phosphorylation status measured in a sample obtained from a healthy mammal, in particular healthy subject.

In a further embodiment the inventive assay method comprises a further step in which proteins that are colocalized with PGRMC1 are determined.

The determination of the phosphorylation status is performed in a further embodiment by a radioactive labelling combined with a gel electrophoresis technique. For instance, the radioactive labelling can be performed as an inverse radio active labelling, preferably with iodine isotopes. Particularly, an inverse radioactive labelling is performed using ¹²⁵I and ¹³¹I isotopes. A suitable gel electrophoresis technique relates to SDS-PAGE (sodium dodecylsulfate polyacrylamidgel electrophoresis), especially to dimensional PAGE (2D-PAGE), preferably two dimensional SDS-PAGE (2D-SDS-PAGE). In a further embodiment, the assay method is based on 2D-PAGE, in particular using immobilized pH gradients with a pH range preferably over pH4 to 9.

The above-mentioned gel electrophoresis techniques may be combined with other protein separation methods, for instance chromatography and/or size exclusion. Furthermore, the assay may comprise suitable detection methods, in particular antibody detection and/or mass spectrometry. A suitable antibody detection can be part of an immuno assay, for example ELISA (enzyme linked immuno sorbant assay) that is preferably part of the inventive assay method.

Regarding possible mass spectrometry techniques, the inventive assay method may comprise the application of MALDI (matrix assisted laser desorption/ionisation) and/or SELDI (surface enhanced laser desorption/ionisation). It is further within the scope of the assay method that resonance techniques, in particular plasma surface resonance, can be applied.

In some cases, it may be preferable to achieve a separation of proteins, in particular of PGRMC1, or isoforms thereof in particular by means of one of the above outlined embodiments, before cleaving the proteins. Such a cleavage may be performed by the administration of enzymes, chemicals or other suitable reagents which are familiar to those skilled in the art. As an alternative, it may be desirable to perform a cleavage before separation of the proteins, in particular of PGRMC1, obtained by the cleavage step followed preferably by measurements of PGRMC1 concerning its abundance and/or degree of phosphorylation.

In a further embodiment, labelled and in particular separated protein spots are visualized by imaging techniques, for instance by the Proteo Tope® imaging technique of the applicant.

Since the difference between the PGRMC1 isoforms under consideration is due to the presence of phosphate residues all methods enabling the detection of subtle or extreme differences in the stoichiometry of phosphate or oxygen atoms in proteins are within the scope of the present invention. In this regard, preferred methods may relate to elemental analysis, measurement of the state of ionisation or differential electrical conductivity. Furthermore, methods enabling the measurement of differences in the stable isotope content of proteins, in particular of chemically modified proteins, or degradation products thereof may be part of the inventive assay method.

The inventors conducted cell culture experiments with stable and transient transfected cells, respectively, using plasmids encoding wild type and mutant mPR proteins, respectively, and observed phenotypes.

For this purpose the MCF7 breast cancer cell line and plasmid pcDNA3.1 expressing a C-terminally tagged PGRMC1 protein were used. The C-terminal tag consisted of three repeats of amino acids from the heamaglutinin protein, generating the protein (PGRMC1-3HA SEQ ID NO: 4).

The inventors observed differences in phosphorylation status between estrogen receptor positive tumours and estrogen receptor negative tumours of the breast. Since the prognosis for estrogen receptor-negative tumours is quite poor, with those tumours exhibiting resistance to treatment and causing high mortality levels, the inventors reasoned that mPR phosphorylation status contributed to the resistance to treatment.

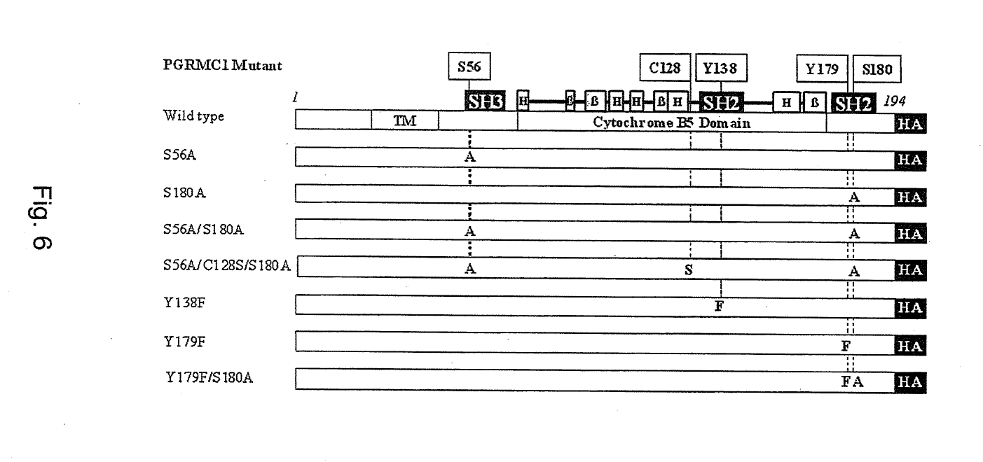

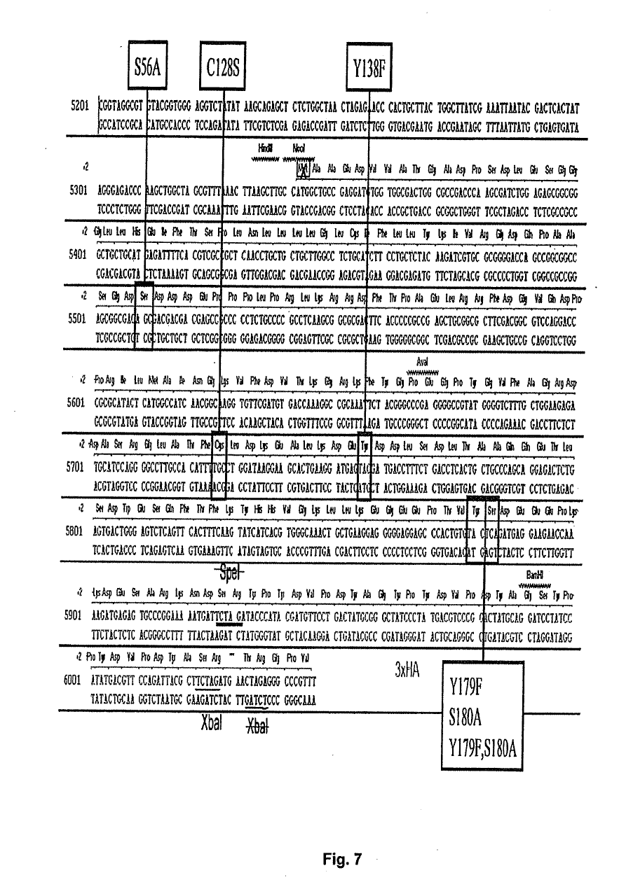

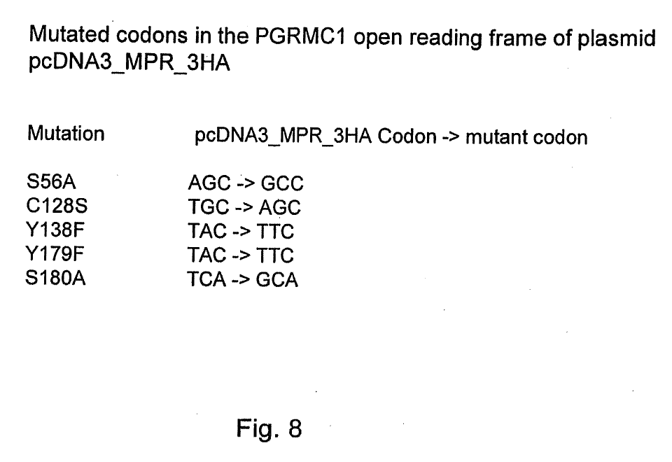

Bioinformatics analysis revealed the presence of potential phosphate acceptor sites in association with the recognition motif sites for SH2 and SH3 domain-containing interaction partners, as described. Accordingly, the potential phosphate acceptor amino acids shown in FIGS. 6 and 7 on the protein surface were mutated to chemically related amino acids that should exert only minimal influence on protein folding. The PGRMC1 open reading frame (ORF) was present in the eukaryotic plasmid expression vector pcDNA3_PGRMC1_{—}3HA. The nucleotide sequence of pcDNA3_PGRMC1_{—}3HA is shown as SEQ ID NO:2. The amino acid mutations that were introduced into the ORF of mPR by site-directed mutagenesis of plasmid pcDNA3_PGRMC_{—}3HA are shown in FIGS. 6 and 7, and the amino acid sequence of the mutated proteins, are shown in SEQ ID NO:5 to SEQ ID NO:11. The codon changes that were introduced to accomplish these mutations are presented in FIG. 8. The same effects are obtainable using PGRMC1 that lacks a hemaglutinin (HA) amino acid tag. Although not all possible mutants are shown in this example in principle all PGRMC1 variants are in the scope of the herein present invention, including all mammalian homologues of PGRMC1.

The present invention is further directed to the use of phosphorylated and/or non phosphorylated progesterone receptor membrane component 1 (PGRMC1), in particular at least one isoform of progesterone receptor membrane component 1 (PGRMC1), as diagnostic marker in diagnosis for diseases associated with aberrant biological phenotypes, wherein the phosphorylation status of the at least one isoform of progesterone receptor membrane component 1 (PGRMC1) is determined and/or estimated.

In a further embodiment the phosphorylation status of PGRMC1 is determined and/or estimated by means of affinity reagents. As affinity reagents may be used any reagent or method that is familiar to a person skilled in the art. Preferred reagents comprise antibodies, aptamers, RNA display, phage display or combinations thereof.

According to an additional embodiment the phosphorylation status of PGRMC1 is determined and/or estimated by means of derivatising the phosphate residues of PGRMC1. Suitable derivatising techniques comprise the incorporation of radioactive atoms into the phosphate residues, elimination reactions and/or Michael additions. The derivatising techniques preferably allow for the detection of original (underivatised) phosphate residues in PGRMC1. The detection may be performed by the aid of fluorescence or surface plasmon resonance.

According to a preferred embodiment of the invention the phosphorylation status is determined and/or estimated by analysis of proteins that are involved, in particular differentially involved, in protein interaction or multi-protein complexes with either phosphorylated or non-phosphorylated at least one isoform of progesterone receptor membrane component 1 (PGRMC1).

Further, the present invention comprises the use of at least one reagent for influencing, in particular increasing or inhibiting, the abundance and/or activity of isoforms of proteins for diagnosis and/or therapy of diseases associated with aberrant biological phenotypes, wherein said proteins are involved, in particular differentially involved, in protein interaction or multi-protein complexes with either at least one isoform of phosphorylated or non-phosphorylated progesterone receptor membrane component 1 (PGRMC1)

According to a preferred embodiment of the invention said diseases, in particular subgroups thereof, comprise cancer, especially breast cancer or prostate cancer, neurodegenerative diseases, infertility, inflammatory, immunological, respiratory, pulmonary diseases, and/or diseases associated with the rate of biological aging or with beneficial or detrimental alterations of the level of the process of autophagy.

The inventors were capable of proving the evidence of three isoforms of PGRMC1. Accordingly, it is in particular preferred to use any combinations of these isoforms as diagnostic markers and/or therapeutic targets for diseases that are associated with aberrant biologial phenotypes.

As a consequence, the invention relates in a further embodiment to the use of phosphorylated and/or non-phosphorylated progesterone receptor membrane components 1 (PGRMC1), in particular at least one isoform thereof, as diagnostic marker and/or therapeutic target for subgroups of diseases that are associated with aberrant biological phenotypes. Preferred aberrant biological phenotypes comprise cancer, preferably breast cancer.

For instance, such subgroups can relate to the abundance of at least one protein, in particular of at least one receptor protein, preferably of estrogen receptor. The estrogen receptor (ER) comprises two types of specific nuclear receptors that are known as estrogen receptor α (ERα) and estrogen receptor β (ERβ). Molecular analysis has proven that ERα, like other nuclear receptors, consists of separable domains responsible for DNA binding (DNA binding domain DBD), hormone binding (hormone binding domain HBD) and transcriptional activation domain. The N-terminal activation function (AF-1) of the purified receptor is constitutively active, whereas the activation function located within the C-terminal part (AF-2) requires hormone for its activity.

As already mentioned, a positive ER status (ER⁺, tumour cells showing abundance of ER) correlates with favourable prognostic features, including a lower rate of cell proliferation and histologic evidence of tumour differentiation. In contrast to that, a negative ER status (ER⁻ tumour cells showing no or at least a decreased abundance of ER) corresponds to substantially poorer disease-free and overall survival probability of the patient. ER status is also prognostic for the site of gross metastatic spread. Besides, tumours with high abundance of estrogen receptor (ER⁺ tumours) are more likely to initially manifest clinically apparent metastasis in bone, soft tissue or the reproductive and genital tracks, whereas tumours with low abundance of estrogen receptor (ER⁻ tumours) more commonly metastasise to brain and liver. Several studies have correlated ERα expression to lower Matrigel invasiveness and reduced metastatic potential of breast cancer cell lines.

The inventors surprisingly found that PGRMC1 was significantly more abundant in breast cancer cells revealing a negative ER status (ER⁻) compared to breast cancer cells revealing a positive ER status (ER⁺). Additionally, the inventors have been the first to prove evidence for different degrees of phosphorylation of PGRMC1 in breast cancer cells differing in the ER status. The alignment of the phosphorylation status of PGRMC1 to subgroups of diseases that are associated with aberrant biological phenotypes, in particular cancer, preferably breast cancer, is advantageous with respect to choice or effectiveness of therapeutic treatments. For instance, regarding patients suffering from breast cancer exhibiting a positive ER status tamoxifen is effective in approximately 50% of the cases. Additionally, the inventors have been the first to detect a wound response signature in breast cancer cells showing a negative ER status (ER⁻) by proteomics and associated this with a degree phosphorylation of PGRMC1.

In a further embodiment of the invention PGRMC1 is derived from mammalian samples, in particular from human samples. In addition, PGRMC1 may be derived from samples that are harvested by biopsy and/or surgical extraction.

In a possible embodiment PGRMC1 is not completely dephosphorylated. Accordingly, it is further within the scope of the present invention that the above-mentioned reagent decreases the degree of phosphorylation of PGRMC1 to a certain extent. In another embodiment, it may be beneficial to maintain the phosphorylation status (degree of phosphorylation of PGRMC1). Therefore, in a further aspect of the invention, at least one reagent may be used as diagnostic marker and/or therapeutic target that maintains the phosphorylation status of PGRMC1, in particular at least one isoform thereof.

Furthermore, the at least one reagent may be a ligand of PGRMC1 that in particular binds to the ligand binding pocket of PGRMC1, or to protein interaction domains of the SH2 and SH3 variety on other proteins which interact with PGRMC1, or with the target sequences for those SH2 and SH3 domains in the PGRMC1 protein, in order to prevent the interaction of PGRMC1 with other molecules, especially proteins, allowing for diagnosis and/or therapy of diseases associated with aberrant biological phenotypes.

The interactions can be observed for example in binding of antibodies directed against PGRMC1, in particular in binding of monoclonal antibody C-262 (StressGen, Victoria, BC, Canada) and in coimmunoprecipitation of gamma aminobutyric acid A (GABA_{A}). Digitonin dependant coimmunoprecipitation of PGRMC1 and caveolin with antisera to caveolin and presence of ITAM motifs (YXX(φ), where φ represents an aliphatic amino acid) support the possible function of PGRMC1 as an adaptor protein involved in regulating protein interactions involved in membrane trafficking, such as endocytosis, exocytosis, or vesicle biology, as well as associated intracellular signal transduction. The presence of a higher order complex of PGRMC1, in particular at least a dimerised form, was indicated by the reduction of disulfide bridges by dithiothreitol (DTT).

In biotinylation experiments it was shown that PGRMC1 was present in an immune pellet precipitated by an antibody directed against a protein denoted as “plasminogen activator inhibitor RNA-binding protein 1” (PAIRBP1) from progesterone-responsive ovarian epithelial cells, and that both coprecipitated proteins in this complex were biotinylated in non-permeabilised cells, indicating that they were present on the outer cell surface. Besides, it was demonstrated by photo-cross linking with UV-sensitive amino acid precursors a physical interaction between co-transfected and affinity tagged PGRMC1 and both “insulin induced gene 1” protein (INSIG-1) and “sterol regulatory element binding protein (SREBP) cleavage-activating protein” (SCAP) in COS7 cells.

In the following the aspects of the present invention are explained in detail:

PGRMC1 interacts with both Insig-1 and Scap1, which are involved in the cholesterol-dependent regulation of the cellular location of sterol regulatory element binding protein (SREBP). In the presence of cholesterol SREBP is retained in the endoplasmic reticulum, whereas it is escorted to the trans-Golgi network and proteolytically processed to release a cytoplasmic fragment which translocates to the nucleus and activates sterol regulatory element (SRE) driven genes. PGRMC1 probably senses levels of cholesterol or its metabolites in the endoplasmic reticulum [18], and therefore regulates this manifestly pleiotropic system.

The presence of conserved YXX(φ) consensus motifs (where φ represents an aliphatic amino acid) in PGRMC1 implicates the protein with an active role in membrane trafficking, since these motifs targets proteins for endocytosis [18;19]. PGRMC1 itself can translocate between endoplasmic reticulum, the plasma membrane, and the cytoplasm under various conditions, and because it acts as a steroid/cholesteroid sensor and contains regulatory protein interaction motifs that are regulated by phosphorylation [18], it is ideally situated to target interacting proteins to various cellular destinations. Therefore a brief consideration of membrane trafficking is appropriate.

Proteins that are newly synthesised within the endoplasmic reticulum may be either retained there, or they may be directed towards the trans-Golgi network. From there, one pathway leads to budding of vesicles that are eventually targeted to the plasma membrane. During the early 1990s it emerged that caveolae were characterized by the presence of scaffold proteins of the caveolin family and were involved in signal transduction and endocytosis/exocytosis processes. Caveolae are small cave-like invaginations of the plasma membrane induced by self-associating caveolin scaffold networks, most of which sequester cholesterol, glycosphingolipids and sphingomyelin as core lipid components to generate a membrane region where signalling complexes can preferentially accumulate and assemble at the surface of the cell. (Cells also contain low cholesterol caveolae. [20]) Membrane microdomains with similar biochemical properties and cell biological functions but lacking caveolin were later defined and became known as “rafts” or “lipid rafts”, of which caveolae membranes form a subclass (for reviews see [20-22]). Proteins on the plasma membrane can be internalised by endocytosis via localisation to invaginating caveolae or larger and more electron dense clathrin-coated pits [23], which reflect the membrane composition of rafts.

Following internalisation by endocytosis, cell surface proteins are precisely sorted in endosomes of the endosomal compartment towards alternative subsequent fates. Some of them are recycled back to the cell surface whereas others are targeted to late endosomes which terminate in lysosomes for proteolytic degradation. Yet other proteins are directed towards multivesicular bodies, where luminal proteins are invaginated into the lumen of the mother vesicle and pinched off to form new vesicles within the lumen of the mother vesicle. The outer membrane of the resulting multivesicular body can fuse with the plasma membrane to release the internal vesicles which are then known as exosomes. This represents a novel pathway of secreting topologically cytoplasmic proteins to the extracellular space of living cells. Ubiquitination of target proteins provides a discriminatory sorting signal during this process. Polyubiquitin groups target cargo proteins to the lysosome, whereas one or more mono-ubiquitin groups direct proteins towards the multivesicular body fate. Ubiquitin-responsive sorting systems containing various proteins with the Ubiqitin Interacting Motif are located in sequential compartments along the secretory and endocytic pathways, and these probably exert their functions in a highly coordinated manner. Furthermore, appropriate ubiquitination of proteins in the late secretory pathway can also target proteins directly to the endosomal compartment without prior cycling to cell surface, or towards the multivesicular body pathway rather than the cell surface. The enrichment of cholesterol lipid rafts at the plasma membrane and in intracellular membrane regions, at least as early in the secretory pathway as the Golgi apparatus, facilitates this sorting process. In fact caveolin is also involved in the direct transport of cholesterol by cytoplasmic diffusion from the endoplasmic reticulum to the cell membrane. Thereby, ubiquitin and cholesterol are central in the control of endocytic sorting, and they are thought to act cooperatively (reviewed in [20;23]). That these processes can manifest severe pathologic aberrations in disease is obvious from the suitability of various E3 ubiquitin ligases, which add ubiquitin to specific target proteins, as cancer targets and biomarkers [24].

PGRMC1 can participate in this system in three ways. Firstly, by affecting cholesterol levels it can modulate the properties of lipid rafts, and thereby exert profound effects on cellular signalling. Secondly, by leaving the endoplasmic reticulum and entering the vesicular trafficking system it can direct interacting proteins to various different locations. Thirdly, its interaction partners can be regulated by differential phosphorylation of its protein interaction sites. Thus, PGRMC1 occupies a nexus function integrating cellular regulation whose influence ranges from the extracellular space, across the cell surface, through various regions of the cytoplasm and into the nucleus.

DCC was originally cloned as a potential tumour-suppressor protein from colorectal cancer, since it was lost by permissive cancers. Netrin receptors are now recognised as belonging to the class of ‘dependence receptors’ which create a cellular state of dependence on their respective ligands by inducing apoptosis in the absence of ligand, and the Netrin system has been recognised as being prominently important for tumour biology [34-36]. The cytoplasmic region of DCC does not contain any catalytic domain but possesses three conserved regions known as P1, P2, and P3. These interact with other receptors to form homo- or heterodimers important for netrin-1 function. The P3 domain is responsible for receptor dimerisation upon ligation which is required for neurite outgrowth. The P3 domain also interacts with the receptor Robo1 which is required for Slit-induced silencing [30;37-40]. Myosin X (Myo X) binds to the P3 domains of Neogenin and DCC, and is involved in the redistribution of DCC on the cell surface into a punctuate distribution. Myo X was required for neurite extension in response to Netrin where it enhanced the formation and extension of filopodia [41].

The absence of Netrin ligand induces self-activation of its receptor and the subsequent proteolytic processing of the receptor triggers apoptotic cell death. Ligand binding blocks the proteolytic processing and the proapoptotic activity of the receptor [31]. In the absence of Netrin, DCC indirectly recruits caspase 9 which activates caspase 3 or an unknown protease that in turn cleaves DCC [34;36]. At least two genetically dissectible signal pathways activated by DCC are conserved with Nematodes, involving Rac and the actin-binding abLIM on one hand and Enabled signalling on the other [42]. Netrin ligation to DCC activates ERK kinases which are recruited to the DCC receptor complex and are ultimately involved in axon migration since specific ERK-kinase inhibitors attenuate migration [43]. In the presence of Netrin, three pathways have been implicated in transducing cytoplasmic signals in mammalian cells, however under what circumstances any or all of these may act in native tissues remains unclear. Firstly, AKT2 may be activated by a protein called APPL (Adaptor protein containing pH domain, PTB domain and leucine zipper motif 1; Also called DIP13α; UniProt ID Q9UKG1) in conjunction with phosphatidylinositol 3-kinase (PI3K). AKT signalling is known to be anti-apoptotic. Secondly, DCC directly binds and inhibits caspase 3, which accordingly cannot cleave DCC. A third mechanism may involve the activation of ERK kinases and concomitant survival signals discussed above (reviewed in reference [36]). Other molecules involved in DCC signalling include a DCC-interacting complex including FAK and FYN kinases through the P3 region, as well as another involving Cdc42, Rac1, Nck1, and Pak1 (reviewed in references [34;37]). Phosphatidylinositol transfer protein-alpha (PITPalpha) binds to both Neogenin and DCC upon ligation of Netrin. This stimulates the binding of PITPalpha to phosphatidylinositol-5-phosphate (PI5P), increasing its lipid-transfer activity and elevating the level of hydrolysis of phosphatidylinositol bisphosphate (PIP2). PITPalpha activity is required for the extension of elongating axons [44]. PIP2 hydrolysis in cortical neurons is mediated by DCC, but not Neogenin or the coexpressed Unc5 family receptor Unc5h2. Netrin-1 induces tyrosine phosphorylation of DCC near the P3 domain which was associated with phospholipase Cgamma tyrosine phosphorylation and activation. Furthermore inhibition of PLC activity attenuates Netrin-induced cortical neurite outgrowth [37].

Caspase activation in response to DCC by Netrin is essential for the attraction to Netrin during axonal guidance, and does not induce apoptosis. This occurs in the axon growth cone and involves the activation of p38 MAPK which locally activates the proteasomal protein degradation pathway that is essential for cytoplasmic reorganisation associated with attraction or repulsion of the growth cone. Caspase 3, but not Caspase 9, is involved in this process.

The response of neurons to Netrin induced by DCC is dependent upon not only the presence of members of the Unc5 and Robo receptor families, but also upon their prior state of activation, and may range from chemoattraction, chemorepulsion, migration, adhesion, or silencing, to apoptosis (reviewed in references [34;45]). For instance, PKC-mediated signalling after binding of Netrin to cells expressing DCC and Unc5A leads to endocytocytosis of Unc5A, changing the cell surface composition of receptors, and causing the axonal response to Netrin to change from growth cone collapse and chemorepulsion to attraction. This demonstrates that the response to Netrin can be dynamically altered over relatively short intervals by protein-directed alterations to cellular activity [46].

Metalloproteases potentiate Netrin-mediated axon outgrowth, suggesting that metalloproteases modulate responsiveness to Netrin by affecting the number of functional receptors on axonal membranes. Normal proteolytic attrition limits the number of functional DCC receptors available to establish active Netrin complexes on extending axons. Netrins intruigingly possess a region with striking similarity to tissue inhibitors of metalloproteases. Therefore metalloproteases are not simply involved in reorganising the extracellular matrix, but also in directly modulate the signals being transmitted through various Netrin receptors [47;48]. Subsequently to cleavage of the extracellular domain by an extracellular protease, the residual ‘membrane stub’ of the DCC receptor, which consists of cytoplasmic and transmembrane domains, can be cytoplasmically cleaved by the presenilin-gamma-secretase complex, which is also responsible for the processing of several type I membrane proteins including Amyloid Precursor Protein and Notch. Gamma-secretase cleavage of cytoplasmic DCC generates a derivative termed DCC-intracellular domain (ICD) which can translocate to the nucleus and that has an intrinsic transcriptional activation domain in the yeast two hybrid system [49], however no transcriptionally activated target genes have been reported. Presenilin cleavage of the intracellular domain terminates signalling from DCC, and this attenuates receptor-mediated intracellular signaling pathways that are critical in regulating glutamatergic synaptic transmission and memory processes [50]. Thus DCC/Netrin signalling governs not only one-time migrational behaviour in neurons, but is also dynamically involved in ongoing essential neuronal functions including those involved in memory formation. Indeed PGRMC1 has been implicated in the response of neurons to progesterone, including survival and dendritic growth, spinogenesis and synaptogenesis [51;52]. Therefore pharmaceutical reagents that affect PGRMC1 function can be employed in the treatment of memory disorders as well as cancers of the nervous system.

Gamma-secretase associates in a cholesterol-dependent manner into lipid rafts of post-Golgi and endosomal identity, whereas most of its substrates are spatially segregated into cholesterol poor and detergent soluble non-lipid raft membranes. Yet gamma-secretase processing of substrates such as DCC and N-cadherin c-terminal fragments that have already been extracellularly proteolysed occurs in non-raft fractions in embryonic brain. Furthermore, signalling caused by regulated intramembrane proteolysis of specific substrates is regulated by the partitioning of active gamma-secretase into cholesterol rich lipid raft domains, and can be perturbed by disruptions to the cholesterol balance [53]. The intimate association of PGRMC1 with cholesterol metabolism and its feedback control has been reviewed [18].

An intracellular pool of DCC exists in vesicles that are targeted to the plasma membrane upon PKA activation. Translocation of DCC depends upon active adenylate cyclase, PKA, and exocytosis. Therefore netrin-1 can increase the level of cell surface DCC via cAMP and PKA, potentiating the response to netrin-1 [54]. PGRMC1 interacts with DCC and has been implicated to be involved in vesicle trafficking [18;19], thereby posttranslationally modulating the availability of its interaction partner DCC (and Neogenin) at the cell surface.

In this context it is pertinent to note that DCC cleavage by caspase 9 to induce apoptosis in the absence of its Netrin ligand requires the localisation of DCC to cholesterol rafts [55]. Thereby differences in PGRMC1 phosphorylation can not only direct the localisation of DCC from intracellular vesicles to the cell surface, but also potentially affect the localisation of DCC in cholesterol-rich lipid-rafts that are required to induce apoptosis.

The Netrin Signalling System in Non-Neurons

The biological processes affected by the Netrin/Neogenin system include a broad range of epithelial morphogenesis and maintenance processes in addition to neuronal guidance and survival [34;56;57]. Netrin is not only a neural attractant but also an angiogenic factor. Neogenin mediates Netrin signaling in vascular smooth muscle cells, whereas another unidentified receptor mediates the proangiogenic effects of Netrin-1 on vascular endothelial cells [34;58]. DCC-dependent vasculogenesis after Netrin exposure is induced via ERK kinases upstream of Endothelial Nitric Oxide Synthase (eNOS), an enzyme from mammalian blood vessels which leads to NO^{•} production in aortic endothelial cells. This in turn induces migration and cell division leading to vessel initiation and extension [59]. The modulation of DCC activity by PGRMC1 is especially interesting in this context because of the involvement of PGRMC1 in the mevalonate pathway leading to cholesterol synthesis and production of prenyl lipids (reviewed by reference [18]). PGRMC1 is explicitly required for cholesterol synthesis [60]. Statins inhibit the enzyme 3-hydroxy-3-methylglutarate-CoA reductase (HMG-CoAR) which is an SREBP-regulated gene that is the rate limiting enzyme in the mevalonate pathway. Statins can enhance NO^{•}-dependent angiogenesis by reducing caveolin-1 abundance and its inhibitory effect on eNOS, and this mechanism operates in tumours [22;61]. Thereby PGRMC1 is able to influence the angiogenic effect of DCC-dependent NO^{•} production by modulating both DCC function and the mevalonate pathway which upregulates caveolin and thereby down-regulates eNOS activity. Since eNOS activity is a critical factor associated with inflammatory diseases [22], reagents directed against PGRMC1 are therefore also suitable for the treatment of inflammatory diseases such as atherosclerosis, as well as cardiovascular diseases.

Skeletal muscle cells undergo terminal differentiation whereby they undergo cell fusion to form polynuclear syncitial myotubes. The Neogenin/Netrin system is involved in the generation of signals that lead to myotube formation, where active Neogenin and Netrin are present in a trans-cellular protein complex containing cadherins [62;63]. Cadherins are involved in cell-cell contacts such as through adherens junctions, which mediate contact growth inhibition by limiting cytoplasmic catenin levels while simultaneously generating survival signals. They are also crucial for correct organization of nascent vessels in angiogenesis (reviewed in reference [64]). Based upon phylogenetically conserved localisation of Netrin to the cell surface of cells that express it, Kennedy proposed a role of Netrin/DCC in the maintenance of cell contacts and intercellular interactions in multiple tissue types as early as 2000 [30]. This is strengthened by the observation that DCC is involved in the regulation of cell adhesion in human colorectal cancer derived HT-29 cells, where it associates with ezrin, a linker of membrane to the cytoskeleton [65]. Loss of this regulation is affected when DCC is deleted in colorectal cancer. Thus the survival signals supplied by these dependence receptors are related to contact growth inhibition and linked cytoskeletal organisation, which represents active supervision of static tissue conditions.

Hemojuvelin (HJV) is a member of the RGM family that interacts with Neogenin. These two proteins are involved in a system involving ferritin that leads to increase in cytoplasmic iron levels of Neogenin-expressing cells [57;66]. This is important since iron levels increase the rate of production of reactive oxygen intermediates, which not only influence cellular redox status but also act as transducers in associated signal transduction [67]. The activation of eNOS in vascularisation has been described above.

The Netrin Signalling System in Breast Morphogenesis

The Netrin/Neogenin system directs the developing mammary terminal end buds, which are highly proliferative structures found at the invading edge of developing mammary glands [68]. The mammary end bud is a dynamic system, whose motility requires the successful integration of systemic and local mammotrophic influences coordinating ductal growth regulation, extracellular matrix remodeling, and cell adhesion in the inner end bud. Stem cells present in the ductule walls can also be induced to initiate new end buds to form branches, both during pubertal development and during pregnancy. This system is under hormonal control, responding to progesterone among other hormones. Netrin is expressed on the extracellular surface or is present in the adjacent extracellular matrix of luminal epithelial cells of the end buds. Neogenin is expressed in the immediately overlying cap cells. Null mutations in either mouse gene cause slower end growth and more disorganised end buds: particularly caused by loss of adhesion between luminal epithelial and cap cells, and apoptosis of the disoriented cap cells [69].

The role of the Netrin/Neogenin system in controlling the invasive behaviours of mammary epithelial cells was further characterised by Strizzi et al. [70] who examined Cripto-1 signalling. Cripto-1 is a member of the EGF family that signals through Nodal, a member of the TGFβ receptor superfamily, to induce Netrin slightly and Neogenin markedly. This is associated with increased proliferation, migration, invasion and colony formation by epithelial cells in 3D matrices, which is accompanied by increased AKT phosphorylation with presumed involvement of PI3K. By contrast, treatment with Netrin alone leads to presumed signalling through the Unc5H Netrin receptor and a concomitant anti-invasive phenotype. A neutralising antibody against Unc5H increased the degree of invasiveness in Netrin-treated cells whereby a neutralising antibody against Neogenin decreased the number of invading cells in an in vitro assay [70]. Therefore increased abundance of Neogenin relative to Unc5H was involved with orchestration of the invasive response to Netrin. The ability of PGRMC1 to modulate the availability or function of Neogenin at the cell surface thereby contributes to the hormone responsiveness of this system.

The effect of constitutive Cripto-1 activation described above resembles the phenomenon of epithelial-mesenchymal transition (EMT) [70]. This is a genetic program of mammalian cells characterized by loss of cell adhesion, repression of E-cadherin expression, and increased cell mobility. EMT is essential for numerous developmental processes including mesoderm formation and neural tube formation. Initiation of cancer metastasis involves tissue invasion, which has many phenotypic similarities to EMT, including a loss of cell-cell adhesion mediated by E-cadherin repression and an increase in cell mobility. EMT has been explicitly linked to the ability of breast cancer cells to enter the circulation and induce metastases [71-74]. The above identifies Neogenin as an orchestrator of EMT in breast cancer. Furthermore, ER− tumours exhibit a basal rather than luminal phenotype accompanied by a higher propensity to seed metastases and poorer patient prognosis [11;75].

Hypophosphorylated isoforms of PGRMC1 were more abundant in ER− tumours (PCT/EP2006/009351) and the phosphorylation sites are present in protein regions that are involved in protein interactions [18]. Therefore PGRMC1 interacts with a different set of cellular proteins in ER− tumours, which explains the different subcellular localisation of PGRMC1 between tumours possessing or lacking the ER (PCT/EP2006/009351). Neogenin interaction with PGRMC1 is obvious from conservation of this biological system with nematode homologues [19]. The ability of PGRMC1 to modulate the availability or function of Neogenin at the cell surface thereby identifies PGRMC1 as a key target for cancer therapy. This carries the further advantage that EMT is a non-essential function for overall survival in adults, and therefore this system is amenable to pharmacological intervention with only moderate sideeffects.

This identifies a biological system whereby breast cells can be induced to exit the dormant Go cell cycle and begin to both invade adjacent tissue and enter the cell division cycle. Both of these processes are deregulated in pathological cancer cells, emphasising that deregulation of the Netrin signalling system is of paramount importance to cancer. Indeed normal breast tissues express higher levels of Neogenin than infiltrating ductal carcinoma [76], strongly implicating perturbations in the Neogenin/Netrin system in tumour neogenesis. Furthermore involvement of PGRMC1 in this mechanism of action is fully concordant with the observed late induction of PGRMC1 in a model of spinal chord injury and wound response, at a time when tissue regeneration was underway [77].

The above demonstrate a plurality of examples of modulation of the Netrin signalling system to control cellular behaviour, and it is remarkable to find avenues for manipulation by PGRMC1 at several levels. In this context it is notable that the PGRMC1-interacting protein PAIRBP1 binds to the cyclic nucleotide-responsive sequence in the Type-1 plasminogen activator inhibitor mRNA [78]. This serine protease inhibitor (serpin) can affect the activity of extracellular proteases that are important for not only extra cellular matrix dissolution and tissue remodelling but also for receptor proteolysis. Furthermore, Netrin/DCC is known to be intimately associated with cAMP signalling [43;79-83], and PAIRBP1 binds to cyclic nucleotide-responsive regulatory sequences in mRNA. PGRMC1 can sequester PAIRBP1, thereby modulating this regulation. Therefore PGRMC1 manipulates the Netrin signalling pathway at various stages, disclosing multiple and previously unrecognised levels at which PGRMC1 and its differentially phosphorylated isoforms can influence the motility and survival of tumour cells. These are biological processes that are extremely relevant to tumour progression and metastasis.

For a more detailed description of the present invention, reference will now be made to the accompanying table and figures:

### Table 1: Pooling Design for ER⁺ vs ER⁻ Cryogenic Whole Tumour Sections

Individual tumours are designated by their tumour bank T registration numbers. Experimental, clinical and histopathological parameters are listed. Eight ER⁺ and eight ER⁻ tumours are grouped into four pools of two tumours each as indicated. Clinical data comprise: tumor status ranging from pT1 (tumor 2 cm or smaller in greatest dimension) to pT3 (tumor >5 cm); Lymph node status from pN0 (no regional lymphnode metastasis) to pN3 (metastasis to ipsilateral internal mammary lymph nodes(s)) and pNx (regional lymph node cannot be assessed); tumour grade from 2 (moderately differentiated) to 3 (poorly differentiated); Histopathological data for ER and PR (0: undetectable, 1-3: weakly positive, 4-7: moderately positive, 8-12: highly positive); and HER2/neustatus (0=negative, positive +1 to +3). Ages for each patient are given in years.

### Table 2: Protein Spots that Contained Multiple Identifications of Individual Proteins as Gene Products

The protein name and number of spots are indicated in the column headings. Approximate estimates for the experimentally observed isoelectric point (PI) and molecular weight (MW) are given for each spot, as are Genbank accession numbers and PMF scores, the nomenclature conventions for which follow FIG. 3.

FIG. 1: 54 cm differential ProteoTope® analysis

The panels show actual images from an inverse replicate labelled ProteoTope® experiment for one sample pair. (A) Analysis of pooled sample ER⁺¹ (ERpos1) from table 1 labelled with I-125, differentially compared with pooled sample ER⁻¹ (ERneg1) labelled with I-131. The lower panels show the signal detected for each isotope, depicted in false spectral colour. The signals for each isotope have been normalised against each other for total relative intensity in the upper dual channel images, where the signal for I-125 is blue, the signal for I-131 is orange, and equal amounts of both signals produces grey or black signal. Two pure sources each of I-131 and I-125, as well as a 50% mixture of both isotopes, are measured on round 2 mm pieces of filter paper placed next to each gel as imaging controls. Cross talk between the signals from each isotope is <1%. The pH ranges of the 18 cm IPGs used for serial IEF are indicated above the panels, and the radioactive iodine isotope signals depicted in each panel are indicated on the right. In this experiment all iodination reactions were performed on 60 μg protein. In the examples shown, the I-125 is signal is systematically stronger in all gels (compare lower panels for individual isotopes). (B). The top panels show the inverse replicate experiment of A, where sample ER⁺¹ is labelled with I-131, and sample ER⁻¹ is labelled with I-125. The bottom panel shows an enlarged portion of a gel image, as indicated. Similar gels were produced for all corresponding differential analyses depicted in table 1.

FIG. 2: Typical example of a synthetic average composite gel of the pH 5-6 analysis, showing spots matched across all gels in the study in this pH range from FIG. 1

The average ER⁺ signal is indicated as blue, the average ER⁻ signal is indicated as orange, and equal intensities of both signals give grey or black pixels. Spot numbers correspond to FIG. 3. Some orange or blue spots that are not numbered (e.g., those labelled ‘X’) were not visible on preparative silver stained tracer gels, and were omitted from the analysis. This image was generated with the GREG software. Labels were added manually.

FIG. 3: Protein spot quantification and identifications for breast cancer samples (whole tumour slices) comparing ER positive and ER negative samples

n.i.=not identified. Genbank Identities are from the NCBI data base version of Apr. 4, 2004. MALDI-TOF peptide mass fingerprinting (PMF) scores are from MASCOT. The average spot fraction for ER⁺ and ER⁻ are given as percent of the normalised total spot volume for each spot (=(ER⁺×100%)/(ER⁺+ER⁻)) across all patient pools based on two colour ProteoTope® analysis for the indicated most significant protein spots. These values were obtained using a least square fit for a model based on all replicates and attributing pool variability as a random effect. The t-test p-value for this model is also given. P-values<0.01 are bold, and p-values<0.001 are designated as such. The bars at the right depict average percent abundance of each protein across the ER⁺ (dark blue) and ER⁻ (light orange) pools as indicated above the column with bars (0%-50%-100%). Error bars show standard error of means. Protein spots between numbers 37 and 38 (indicated by a grey field) are not presented, having failed to meet selection criteria of either abundance difference ratio of 1.5 or significance at the 5% level.

FIG. 4: PGRMC1 immune histochemistry in ER⁺ and ER⁻ tumours

The rabbit polyclonal anti-PGRMC1-specific signal (green) is associated with diffuse cytoplasmic staining in ER⁺ tumours (A-C), whereas anti-PGRMC1 signal exhibits increased localised concentration to specific extra-nuclear sub-cellular locations in ER⁻ cells (D-F). The dark purple colour is hematoxylin counter-staining of nuclear chromatin. The 10 μm scale bar is shown in each panel. A and B show the PGRMC1 staining pattern of two different tumours, while C shows an enlargement of the framed region from B, as indicated. The same relationship applies to D,E and F. The rabbit polyclonal antiserum was a gift of F. Lösel (University fo Heidelberg).

FIG. 5: Differential quantification of phosphatase treated and control samples

(A-F) Inverse replicate ProteoTope® images of the gel region containing three spots of PGRMC1: spots 38, 52, and 62 from FIG. 3. Image conventions follow FIG. 1. (A) The phosphatase treated sample (+SAP) is labelled with I-125 (blue colour), and the mock incubation control (−SAP) is labelled with I-131 (orange colour). Spot numbers are indicated, and are applicable to all panels. (B) The inverse replicate experiment to A. (C) I-125 labelled +SAP is analysed against I-131 labelled untreated raw control sample (raw). (D) The inverse replicate experiment to C. (E) I-125 labelled -SAP is analysed against I-131 labelled raw control. (F) The inverse replicate experiment to E. (G) Quantification of the differential ratio of signal intensities from sample 2/sample 1 for each of the spots from the gels shown in A-F. The identity of sample 1 and sample 2 for each comparison are shown at the right hand side of the panel, with corresponding colour coding. The ratio of signal for control and treated samples increases in a phosphatase-dependent manner, consistent with spots 38 and 62 representing phosphorylated isoforms of spot 52.

FIG. 6 This figure shows the PGRMC1 proteins expressed from stably transfected plasmids as described in PCT/EP2006/009351. Amino acid numbering is according to the PGRMC1 amino acid sequence of human Uniprot entry O00264, which does not include the initiator methionine. Uniprot Q6IB11 corresponds to the same sequence with initiator methionine included, where mutated human PGRMC1 amino acids would be numbered as Ser57, Cys129, Tyr139, Tyr180, Ser181, etc.

FIG. 7 The sequence of wild type PGRMC1 (also known as mPR) is indicated, as well as location of site directed mutations of FIG. 8.

FIG. 8 Site directed mutations introduced into the open reading frame of plasmid pcDNA3_MPR_HA to generate the amino acid mutations indicated. The positions of each codon within the PGRMC1 open reading frame are shown in FIG. 7.

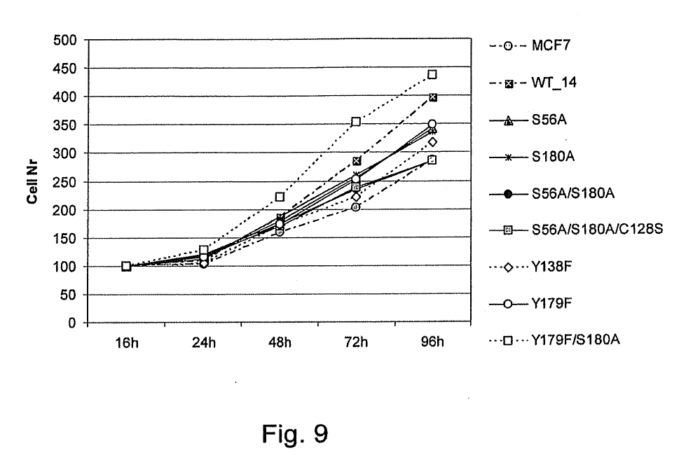

FIG. 9 Growth kinetics of PGRMC1 mutants. 5000 cells were plated into 96 well plate in triplicates and incubated at 37° C./5% CO2 in RPMI with 10% FCS. At indicated time points supernatant was decanted and ATP-Assay was performed. This figure shows that the Y179F/S180A mutant proliferates faster than other cells in the assay. The error bars (standard error) are shown, yet are in all cases smaller than the size of the symbols of plotted points.

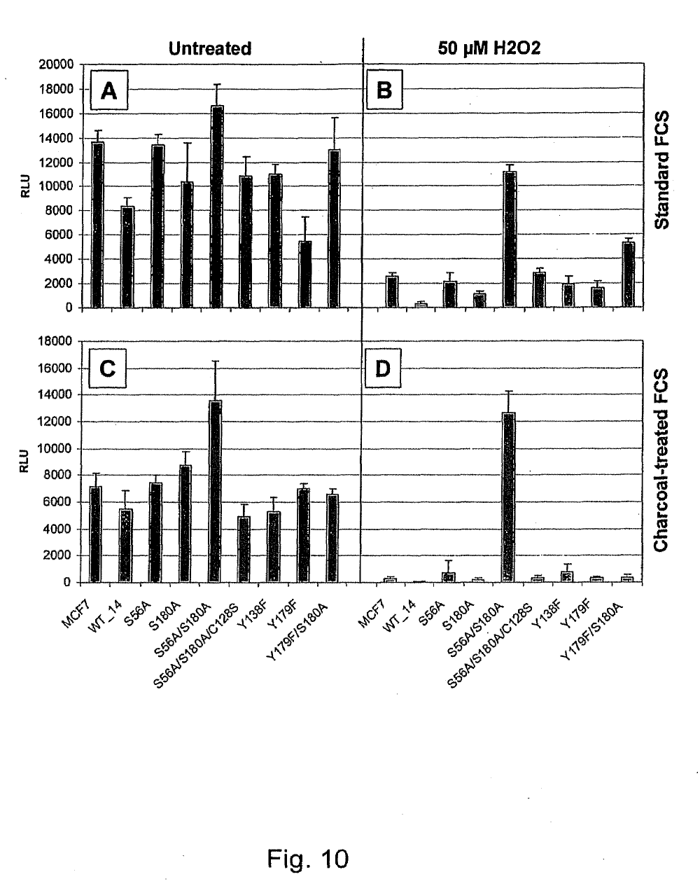

FIG. 10 Effects of PGRMC1 mutants on cell viability after H2O2 treatment. A) Cell lines were grown in RPMI with 10% FCS. B) Cells grown in RPMI with 10% FCS with addition of 50 μM H2O2. C) The analogous experiment to A, performed using charcoal-treated FCS to remove steroids and cholesterol. D) The analogous experiment to C, with addition of 50 μM H2O2. This figure shows that S56A/S180A and Y179F/S180A are resistant to H2O2-induced cell death in medium containing normal serum, whereas only S56A/S180A is resistant in medium containing charcoal-treated serum, which removes hydrophobic molecules such as steroids. RLU=Relative light units, whereby ATP is detected by a fluorescence reaction.

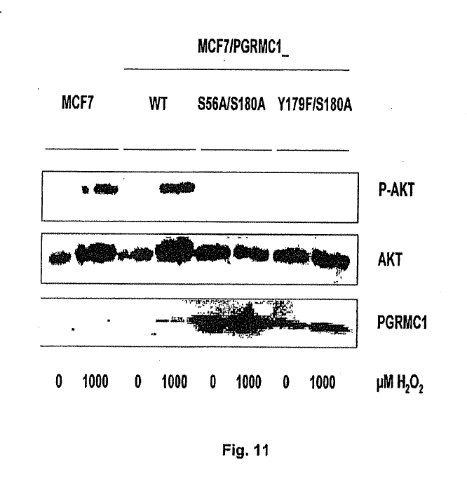

FIG. 11 PGRMC1 influences phosphorylation of AKT after H2O2 treatment. The figure shows a western blot of equal total protein amounts (10 μg/lane) from cell extracts fo the indicated cells. Antibodies employed were specific for phosphorylated AKT (top), for the polypeptide backbone of AKT (middle), or for the HA-tag on the respective exogenously expressed PGRMC1 constructs.

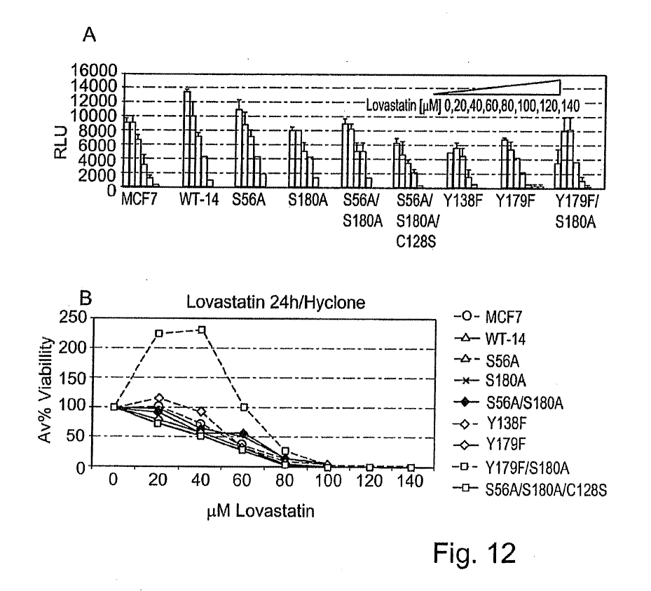

FIG. 12 PGRMC1 S180A abrogates Methyl-β-cyclodextrin-induced death.

This figure shows that the response to Methyl-β-cyclodextrin (Methly-beta-CD) is different than to H2O2. All PGRMC1 mutants except S180A induce enhanced cell death. Methly-beta-CD was purchased from Sigma (#C4555) and administered as described [53]. 5000 cells were plated into 96 well plate in triplicates and incubated at 37° C./5% CO2. On day two medium was changed to RPMI/Hyclone charcoal-treated FCS. On day 3 cells were washed with PBS and incubated with 5 mM Methyl-beta-Cyclodextrin in RPMI/Hyclone charcoal-treated FCS for 2 h. Then the ATP-Assay was performed.

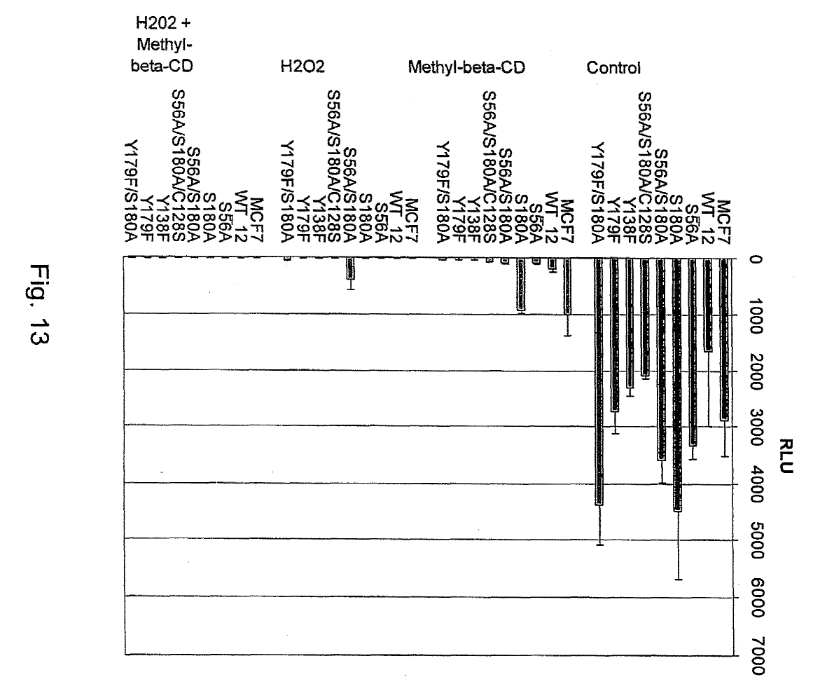

FIG. 13 PGRMC1 modulates the mevalonate pathway. A. Replicate values ±standard error of cell survival assays under the indicated concentrations of lovastatin for the PGRMC1 mutants. Cells are grown in RPMI medium with charcoal-treated FCS which is depleted in cholesterol. RLU=relative light units. B) Average viability of mutants from A, normalised to growth conditions in the absence of lovastatin (100% at zero concentration). Lovastatin was purchased from Sigma (#M2147) and administered as described [87]. 5000 cells were plated into 96 well plate in duplicates and incubated at 37° C./5% CO2. On day 2 cells were incubated with increasing levels of lovastatin for 2 days, followed by cell harvest and performance of the ATP-Assay.

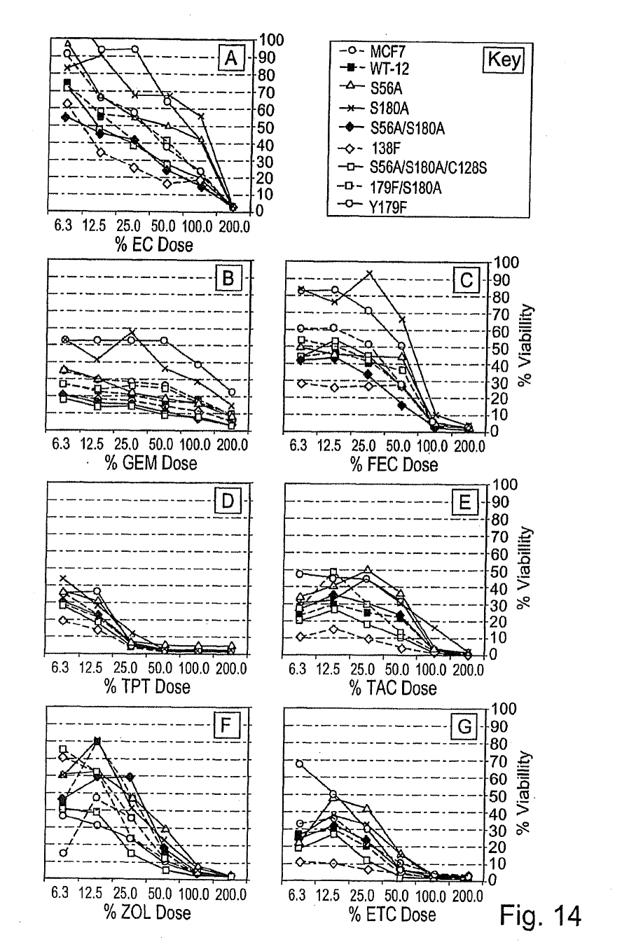

FIG. 14 Response of cells expressing PGRMC1 mutants to cytostatic reagents. The clinical cytostatics used were: A) EC: Epirubicin/Maphosphamid; B) GEM: Gemcitabine; C) FEC: 5-Fluoruracil/Epirubicin/Maphosphamide; D) TPT: Topotecan; E) TAC: Taxol/Adriamycin/Maphosphamide; F) ZOL: Zoledronic acid (Zometa); G) ETC: Epirubicin/Taxol/Maphosphamid. Concentrations are given as percent respective clinical Test Drug Concentration (TDC) dosage. The key shows the identity of mutants from FIG. 1-FIG. 3. 5000 cells were plated into 96 well plate in duplicates and incubated at 37° C./5% CO2 for 7 days in RPMI with 10% FCS. After 7 days the cells were harvested and ATP-Assays for viability were perfomed (on day 10 of the experiment). Each point is the average of replicate determinations. The 100% TDCs for the cytostatics were 5-Fluoruracil: 170 μM; Epirubicin: 0.9 μM; Gemcitabine (Gemzar): 219 μM; Taxol: 15.9 μM; Topotecan: 1.5 μM; Cyclophosphamid/Maphosphamid: 11.5 μM; Zoledronic acid: 34.5 μM; Doxorubicin/Adriamycin: 0.9 μM.

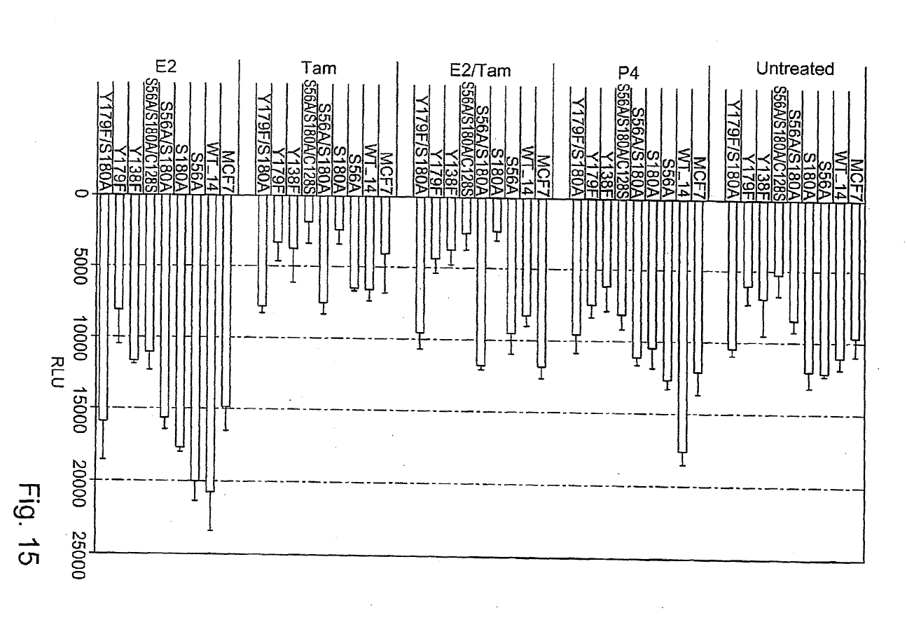

FIG. 15 Effects of hormone treatment on PGRMC1 mutants. 5000 cells were plated into 96 well plate in triplicates (Tamoxifen only in duplicates!) and incubated at 37° C./5% CO2. On day two medium was changed to RPMI with 10% charcoal-treated Hyclone FCS and hormones/anti-hormone were added on day three. The indicated treatments were P4: progesterone (10⁻⁷ M); E2: estrogen (10⁻⁹ M); Tam: tamoxifen (10⁻⁸ M). After growth in the presence of hormones for 5 days an ATP-Assay was performed.

### MATERIALS AND METHODS

Patients and tissue samples. Primary breast cancer specimens were obtained with informed consent from patients, who were treated at the Department of Gynecology and Obstetrics, University Hospital Tübingen (Ethikkommission Med. Fakultät AZ.266/98). Samples were characterized and collected by an experienced pathologist. After removal of breast tumour from the patient, the tissue samples were embedded in O.C.T. compound (Leica), then snap frozen in liquid nitrogen within 15 minutes of tumour removal, and stored at −196° C. in a tumour tissue bank. Sample collection was approved by an ethics committee and by the patient. Tumour data were stored in an Oracle-based database according to practices approved by the Institute of Electrical and Electronics Standards Association (IEEE-SA). Clinical information was obtained from medical records and each tumour was diagnosed by a pathologist, according to histopathological subtype and grade. The tissue quality of each tumour was verified by measuring RNA integrity from one or more slices with an Agilent 2001 Bioanalyser. Tumours lacking sharply distinct 18S and 28S ribosomal RNA bands were excluded from the study. ER, PR and HER-2/neu status for each tumour were routinely determined by immunohistochemistry.

### Preparation of Cryosections

Tumour samples were selected using the database, removed from the tissue bank on frozen CO₂ and transferred to a cryotome (Leica) at a temperature of −23° C. Cryogenic sections (10 μm) were subsequently sliced, placed on SuperFrost+-slides (Multimed) and stored at −80° C. until further use. For immunopathologic characterisation by an experienced pathologist one section was stained with hematoxilin/eosin.

### Proteomics Analysis

ProteoTope® analysis was performed essentially as described (Neubauer H, Clare S E, Kurek R, Fehm T, Wallwiener D, Sotlar K, Nordheim A, Wozny W, Schwall G P, Poznanovic S, Sastri C, Hunzinger C, Stegmann W, Schrattenholz A, Cahill M A. 2006. Breast cancer proteomics by laser capture microdissection, sample pooling, 54-cm IPG IEF, and differential iodine radioisotope detection. Electrophoresis. 27:1840-52.). Frozen tumour sections of 10 μm were lysed directly into SDS buffer, separately iodinated in inverse replicated with each of I-125 and I-131, and separated by 54 cm daisy chain IEF-IPG after sample pooling as described in Table 1. Aliquots of each sample were each iodinated by either ¹²⁵I or ¹³¹I, respectively, using approximately 6 MBq of each isotope per 3.6 μg pooled sample aliquot under identical chemical conditions in a reaction volume of 25 μL by the iodogen method as described. Radioactive iodine was purchased from Amersham Biosciences (Freiburg). 2D-PAGE was performed using 18 cm commercial immobilised pH gradients (IPGs) in serial 54 cm IPG-IEF over pH 4-9 (pH 4-5; pH 5-6; pH 6-9) that were run in the SDS-PAGE dimension as 3×18 cm IPGs in a Hoefer ISO-DALT.

### Shrimp Alkaline Phosphatase (SAP) Analysis

Cryogenic slices from 6 patients (30 slices T433, 40 slices T443, 40 slices T469, 40 slices T470, 35 slices T623, 30 slices T640) were each extracted with 200 μL aliquots of SAP-dephosphorylation buffer (50 mM Tris pH 8.5, 5mM MgCl₂, 0.25% CHAPS, supplemented with 1× EDTA-free Complete protease inhibitor cocktail from Roche). This precooled buffer was added directly on ice to the frozen slices in eppendorf tubes and the tissue was mechanically homogenised using a plastic pellet pestle. Tubes were vortexed and incubated for 30 min at 4° C., followed by centrifugation for 15 min at 14 000×G at 4° C. Supernatants were collected together and the protein concentration was assayed by the BCA method as described. The yield was approximately 4 mg of protein. 30 Units of SAP in 30 μL were added into 800 μg of protein in 400 μL of in SAP-Dephosphorylation buffer, followed by mixing and incubation for 16 h at 37° C. In parallel a mock incubation control was performed on 800 μg of protein in the same buffer without the addition of SAP, and containing the following phosphatase inhibitors: activated vanadate, sodium fluoride, and sodium glycerophosphate at final concentrations of 1 mM, 5 mM, and 5 mM respectively. The incubation was performed in parallel at 37° C. for 16 h. Following incubation the proteins were frozen at −80° C. A non-incubated raw lysate control containing 800 μg of protein in 400 μl of SAP buffer was frozen at −80° C. without additions or incubation. Frozen protein mixtures were thawed, precipitated, and resuspended at 1 μg/μl in boiling 0.1M Tris, 2% SDS, pH8.5. 60 μg of protein were then used for iodination with each of I-125 or I-131 as described. Differential inverse replicate ProteoTope analysis was as described above for 54 cm daisy chain IPG-IEF after rehydration loading overnight to the pH 5-6 IPG.

### Protein Identification by Mass Spectrometry

Protein identification is based on different mass spectrometric methods: an automated procedure that allows a very quick and reliable identification of higher abundant proteins (peptide mass fingerprinting with MALDI-TOF-MS) but also allows the identification of very low abundant proteins with more time consuming procedures (LC-ESI-IonTrap-MS/MS, or MALDI TOF-TOF). Briefly, gel plugs of selected protein spots are excised and the proteins contained in the gel plugs are digested using trypsin. The resulting solution is analysed first with a high throughput peptide mass fingerprint procedure based on MALDI-TOF-MS. For those spots where only ambiguous identification was achieved, a fragment ion analysis based on MALDI TOF-TOF or LC-ESI-IonTrap-MS/MS was added. A detailed description of typical MALDI-TOF-MS procedures has been published (Vogt J A, Schroer K, Holzer K, Hunzinger C, Klemm M, Biefang-Arndt K, Schillo S, Cahill M A, Schrattenholz A, Matthies H, Stegmann W. 2003. Protein abundance quantification in embryonic stem cells using incomplete metabolic labelling with 15N amino acids, matrix-assisted laser desorption/ionisation time-of-flight mass spectrometry, and analysis of relative isotopologue abundances of peptides. Rapid Communications in Mass Spectrometry, 2003; 17:1273-1282).

### Database Searching

For the identification of the proteins the peptide masses extracted from the mass spectra were searched against the NCBI non-redundant protein database (www.ncbi.nlm.nih.gov) using MASCOT software version 1.9 (Matrix Science, London, detailed description can be found at http://www.matrixscience.com/).

### Site-Directed Mutagenesis

Mutations of specific amino acids in pcDNA3.1-PGRMC1-3HA were generated according to standard methods by commercial service providers.

### Cell Culture Experiments

### Stable Transfections

MCF-7 cells stably transfected with PGRMC1 and mutants, respectively, were established. 5 μg of expression plasmid pcDNA3.1 containing hemaglutinin (HA)-tagged PGRMC1 WT or HA-tagged mutants S56A, S180A, S56A/S180A, S56A/C128S/S180A, Y138F, Y179F or Y179F/S180A were transfected into MCF-7 breast cancer cells. For transfections a transfection device and transfection kits from AMAXA Biosystems were used (Gaithersburg, Md., USA) according to the manufacturer's recommendation. 2×10⁶ cells were transfected with circular plasmids and plated with RPMI-Medium for 24 h. Then Medium was changed to RPMI complete medium containing 60 μg/ml hygromycin B and cells were cultured for 2 weeks for selection of stable integration events. After two weeks single colonies had formed and limiting dilution assays were performed to select for colonies grown from a single cell. To that aim colonies were trypsinized, counted and diluted in two-fold dilutions.

### Transient Transfections

MCF-7 breast cancer cells were transfected with 5 μg of pcDNA3.1 containing HA-tagged PGRMC1, tagged mutant A, B, C, or tagged mutant D. The AMAXA transfection system (see above) was used according to the manufacturer's recommendation. After transfection of 2×10⁶ cells the cells were split into 3 wells from a 12-well plate (3.2 cm²). Cells were grown for 24 h. Then medium was changed to RPMI without phenol-red, 5% Hyclone stripped FCS and 1% Penicillin/streptomycin to starve cells for hormones contained by normal FCS. Cells were incubated for 24 h. Afterwards cells were washed with PBS, trypsinized and counted. Cytospins were prepared using 5×10⁵ cells per slide.

### Immune Fluorescence

Cytospins were air dried overnight and then fixed with 0.05% formalin, washed with PBS, treated on ice with PBS/0.1% Triton for 15 min and washed again with PBS. To avoid background labelling, cytospins were blocked with 10% normal serum according to the species of the secondary antibody (here: goat). After removal of the block primary anti-HA-specific antibody (rabbit, Santa Cruz) was applied in a 1:100 dilution in antibody diluent (Dako Norden A/S, Glostrup, Denmark) and incubated for 1 h in a humid chamber at room temperature. The cytospin was washed with phosphate buffer (phosphate buffered saline, PBS) once and then secondary goat anti-rabbit-Alexa Flour 594 antibody (Molecular Probes) was used to detect the primary antibody. Anti cytokeratin antibody was analogously visualised by fluorescein isothiocyanate (FITC). DNA staining was by DAPI (4′,6′-diamidino-2-phenylindole). After 30 min in the humid chamber the cytospin was washed twice with PBS. The cytospins are not allowed to dry. For staining the nucleus VECTASHIELD® Mounting Medium (Vector Laboratories Inc., Burlingame, Calif., USA) was used for embedding the cytospins under a coverslip. Fluorescence was detected using a Metafer4 fluorescence scanning microscope (MetaSystems GmbH, Altussheim, Germany) and the accompanying “Isis” software provided by MetaSystems.

### Immunoprecipitation (IP)

MCF-7 breast cancer cells were transfected by Lipofection with 5 μg of pcDNA3.1 containing HA-tagged PGRMC1-wt, tagged mutant S56A, S180A, S56A/S180A, or tagged mutant S56A/C128S/S180A. After transfection of 2×10⁶ cells they were cultured in RPMI-Medium with 5% FCS. For IP the cells were trypsinized, counted and cell pellets were snap frozen and stored at −80° C.

For IP the pellets were lysed in lysis buffer (M-Per Mammalian Protein Extraction Reagent+Halt Protease Inhibitor Cocktail Kit, PIERCE) and pre-clearance was performed with Protein A Sepharose CL-4B (Amersham) beads (preincubated with rabbit normal serum) for 1 h at 4° C. Beads were separated by centrifugation and stored at −80° C. To affinity purify the HA-tagged PGRMC1 variants these lysates were further incubated with Protein A Sepharose CL-4B preincubated for 1 h at room temperature with polyclonal rabbit anti-HA-antibody (Santa Cruz). Incubation was performed for 16 h at 4° C. in a rotating tube. Then, beads were separated by centrifugation and washed twice with ice cold lysis buffer.

For PAGE gel-loading buffer/mercatoethanol was added to beads and the final supernatant, heated to 95° C. for 5 min and loaded on a 10% polyacrylamide gel. After separation of the proteins, western blot was performed onto nitrocellulose membrane. Transfer was controlled by Ponceau-red staining visualizing protein bands. After destaining the membranes were blocked for 16 h at 4° C. with 5% milkpowder/0.05% Tween. The next day membranes were incubated with mouse monoclonal anti-DCC antibody (1:20) (Biozol/Abcam) for 2 h at RT followed by biotinylated anti-mouse IgG antibody (Vecta Stain). For detection, membranes were incubated with streptavidin/HRP complex (DAKO) for 2 h at RT followed by enhanced chemiluminescence (ECL) treatment and measurement of chemiluminescence using a Lumiimager (Roche). As a loading control, membranes were incubated with rabbit polyclonal Actin I-19 antibody (Santa Cruz).

In PCT/EP2006/009351 an expression plasmid encoding the human PGRMC1 open reading frame with a C-terminal influenza virus hemaglutanin (HA) epitope tag was used as a template to construct specific directed amino acid mutations in PGRMC1 (FIG. 6-8). The construction of PGRMC1 plasmids and the establishment of individual subclones expressing each PGRMC1 variant have been described (PCT/EP2006/009351). The methods, plasmids, mutations in PGRMC1 and PGMRC1 variants disclosed in PCT/EP2006/009351 are hereby incorporated by reference. Same applies to all other cited references which are hereby incorporated into this document, too. Stable transfectants of the breast cancer cell line MCF7 were obtained expressing all mutants. MCF-7 cells were maintained in RPMI medium containing 10% fetal calf serum (FCS), 2 mM L-glutamine, and penicillin and streptomycin. PGRMC1 expression in all cell lines was confirmed by Western blot using an HA-specific monoclonal antibody [84]. Here the effects of those mutations on the response of cells to various stimuli are analysed.

We estimated cell viability using an assay which utilized the fluorometric determination of ATP levels as an indicator of cell viability. The assay for cell viability was performed using the ATP-TCA kit (TCA-100; DCS Innovative Diagnostik Systeme, Hamburg, Germany) as follows: cells were incubated with 50 μl tumor cell extraction reagent and 50 μl RPMI-medium for 30 min at RT. Then 50 μl supernatant was transferred into a 96 well plate and 50 μl chemoluminescence reagent was added. Chemoluminescence was quantitated using a luminescence reader (Berthold, Germany). The fluorometric readout is presented in relative light units (RLU) in the figures, whereby higher RLU values are associated with higher ATP levels and thereby higher levels of cell viability.

First we measured whether any of the mutations in PGRMC1 affected the kinetics of growth in culture of the cell lines. The Y179F/S180A mutant proliferated more quickly than other cell lines as also did WT PGRMC1 to a lesser degree FIG. 9. This indicates that tyrosine phosphorylation of Y179 can exert an inhibitory effect on proliferation, as can serine phosphorylation of S180.

Expression of PGRMC1 in MCF-7 cells sensitized the cells to death following long-term/low dose or short-term/high dose treatment with hydrogen peroxide. Cell death did not occur through a typical apoptotic mechanism and corresponded with hyperphosphorylation of the AKT kinase protein [84]. Cells expressing all PGRMC1 proteins in our panel of mutants exhibited comparable growth in cell culture medium containing normal fetal calf serum (FCS) as the parental MCF7 cells (FIG. 10A). Treatment of MCF7 cells with 50 μM H2O2 reduced cell viability, and exogenous expression of the wild type PGRMC1 protein (designated WT_{—}14 in the figures) further exacerbated this reduced viability (FIG. 5B; compare MCF7 with WT_{—}14) which was consistent with the expected situation as reported [84]. Notably, the viability of the S56A/S180A mutant, and to a lesser extent of the Y179F/S180A mutant, were considerably greater than the viability of MCF7 cells, or cells expressing the WT PGRMC1 protein (FIG. 10B). This trend was also observed at 100 μM H2O2 (data not shown). This result is notable because both of these PGRMC1 mutants produced unusually low numbers of surviving colonies in one round of stable selection (PCT/EP2006/009351).

An experiment was performed with replicate design, but using charcoal/Dextran-treated 10% FCS (Hyclone SH30068). Charcoal-stripping removes steroid hormones and other components from the FCS. All cell lines were able to grow in this medium (FIG. 10C), however the degree of viability was less in the presence of H2O2 for all cells with the exception of the S56A/S180A double mutant (FIG. 10D). Therefore charcoal-treatment of FCS removes some component which is necessary for the survival of PGRMC1 mutant Y179F/S180A, but not for S56A/S180A. Cysteine 128 was essential for this vital function of the S56A/S180 mutant, and it is most probable that dimersiation via a cystine-mediated disulfide bond [18] is required for the rescuing function. Taken together, the growth advantage of the Y179F/S180A mutant translates into a more robust response to H2O2 stress, however this requires some hydrophobic component (such as cholesterol) removed by treatment of FCS with charcoal. The S56A/S180A is able to circumvent H2O2-mediated cell death even when growing in charcoal-treated FCS.

H2O2-induced cell death of MCF7 cells and MCF7 cells expressing exogenous PGRMC1 was associated with phosphorylation of AKT, as reported in the literature [84], therefore we examined AKT phosphorylation of these mutants in FIG. 11. Strikingly, neither of the mutants that survived H2O2 treatment in FIG. 10B exhibited phosphorylation of AKT upon H2O2 treatment, even at 1 mM levels (FIG. 11). Therefore these PGRMC1 mutants are able to exert profound effects on the intracellular signal transduction pathways activated in response to external stimuli, strongly supporting a role for differentially phosphorylated PGRMC1 in the biology of breast tumours.

Because PGRMC1 has been implicated with a regulatory role in cholesterol metabolism, we assayed the effects of treating the cell lines with methyl-β-cyclodextrin (Methly-beta-CD), a drug known to selectively deplete biological membranes of cholesterol (FIG. 13). Whereas the S56A/S180A mutant was once more resistant to H2O2, the expression of all PGRMC1 mutants except S180A drastically reduced cell viability relatively to MCF7 control cells. This demonstrates that PGRMC1 induces a biological response in the absence of cholesterol that compromises cell viability. Serine 180 of PGRMC1 is required to induce the biological effect which kills cells in the absence of cholesterol. Therefore its phosphorylation is probably responsible for the relocalisation of PGRMC1 to a cellular compartment where the biological effect is propagated, possibly from the endoplasmic reticulum to the Golgi-apparatus. Depletion of cholesterol combined with H2O2 treatment was extremely toxic to all cells (FIG. 13). These data are compatible with PGRMC1 monitoring cholesterol levels and inducing a biological response. Furthermore, phosphorylation of Ser180 is involved in the response to depleted cholesterol levels.

At low levels of cholesterol the SREBP protein is escorted from the endoplasmic reticulum to the Golgi-apparatus, where it is proteolytically cleaved to generate the SREBP transcription factor. This induces sterol response element (SRE)-driven genes including HMG-CoA-Reductase which activates the mevalonate pathway and enhances cholesterol synthesis (see [18] for references). Therefore we assayed the effect of lovastatin on the different mutants. Statins are inhibitors of HMG-CoA-Reductase, a rate limiting enzyme in the mevalonate pathway [85]. While the viability of all cell lines was reduced by high levels of lovastatin, the PGRMC1 Y179F/S180A exhibited improved viability at lower concentrations of lovastatin. The Y138F mutant also exhibited a slight trend in this direction, although it was not significantly different than the other clones (FIG. 12). Interestingly the Y179F/S180A mutant exhibited somewhat lower ATP levels in the absence of lovastatin (FIG. 12A), perhaps reflecting constitutive activation of the mevalonate pathway which has some negative effect on viability. Low concentrations of Lovastatin alleviated this effect, elevating ATP production and producing a relative increase in cell survival, whereas higher concentrations of Lovastatin were fatal. Y138F exhibits a slight trend in the same direction. Both of these mutations remove the phosphate acceptor of an SH2-domain target sequence, indicating that tyrosine phosphorylation of PGRMC1 Y179 (and perhaps also Y138) is necessary for interaction with other proteins that mediates repression of the mevalonate pathway.

Next we investigated the potential of PGRMC1 mutants to modulate the survival of cells in response to cytostatic reagents commonly used in chemotherapy (FIG. 14). Strikingly, the mutants Y179F and S180A were often among the most viable cells in these assays, especially for treatment with Epirubicin/Maphosphamide (EC), Gemzitabine (GEM), and 5-Fluoruracil/Epirubicin/Maphosphamide (FEC). For the former two more than 30% of cells were viable even at 100% of the clinically employed concentration (FIG. 14A-C). These treatments consisted of Gemcitabine (a nucleotide analogue that interferes with DNA replication) and Epirubicin (a DNA intercalator that interferes with DNA replication) and Maphosphamide (an alkylating reagent). The FEC treatment also include 5-fluorouracil, another nucleotide analogue. These data provide insight into the mechanism of cell death evasion by PGRMC1, as well as indicating that certain classes of chemotherapeutic reagents will be more effective than others for different tumours containing differentially phosphorylated PGRMC1 protein. This emphasises the diagnostic utility of PGRMC1 phosphorylation.

Crudden et al. have demonstrated that depletion of PGRMC1 from cells by RNAi increases the susceptibility to chemotherapeutic reagents. Furthermore exogenous overexpression of PGRMC1 increased sensitivity to the DNA intercalating agent doxorubicin and the topoisomerase-I modulator camptothecin [86], which induces single strand DNA nicks transcribed genes. It has been suggested that modulation of cytochrome P450 enzyme activity may be responsible for altering the cytotoxicity of cytostatics. Our results demonstrate that the phosphorylation of PGRMC1 is crucial for the response to cytostatic agents employed in chemotherapy, yet the response is mediated by different mutants than the response to either cholesterol depletion or H2O2, clearly identifying PGRMC1 as a multi-functional protein which affects many different cellular responses. There is clearly more to the PGRMC1 response than cytochrome P450 regulation alone. Furthermore, the dynamic reversal of the state of phosphorylation provides the cell with an avenue to regulate these responses via PGRMC1, identifying this protein as a key therapeutic target molecule. Indeed, ER− tumours differ in the phosphorylation status of PGRMC1, and exhibit worse response to treatments including chemotherapy as discussed above.

Finally, we assayed the hormone dependence of various mutants, for which the results are shown in FIG. 15. MCF7 cells express the classical (cytoplasmic) estrogen and progesterone receptors. Progesterone (P4) exerted a marginal proliferative effect on the PGRMC1-WT cell line relative to other cells, yet generally the viability of cells growing in the presence of charcoal-treated FCS was not affected greatly by progesterone. As expected, the viability of cells was improved when estrogen (E2) was added to the RPMI medium containing only charcoal-treated FCS, whereas addition of tamoxifen reduced viability. Tamoxifen (Tam) competes with estrogen for the ER to inhibit the transcription of estrogen-responsive genes, and estrogen could not rescue the impaired viability of cells in the presence of tamoxifen, as expected.

Surprisingly, whereas the expression of exogenous wild-type PGRMC1 did not markedly affect the response of MCF7 cells to Tamoxifen, several of the PGRMC1 mutants greatly reduced cell viability in response to tamoxifen with or without the presence of estrogen. Clone S180A proved to be highly susceptible to Tamoxifen, whereas clone S56A/S180A exhibited resistance to that reagent which was comparable to the wild-type PGRMC1 control or to native MCF7 cells. The reaction profile across the different mutants in FIG. 15 demonstrates that the phosphorylation of PGRMC1 is required by MCF7 cells to survive in the presence of tamoxifen. Furthermore the requirement for multiple phosphorylation sites indicates that the coordinated phosphorylation and dephosphorylation of PGRMC1 is required for this protective phenotype. Cysteine 128, implying a redox-mediated step, was also essential for the ability to remain viable in the presence of tamoxifen as apparent from comparison of the S56A/S180A and S56A/C128S/S180A mutants in FIG. 15.

These results unambiguously identify PGRMC1 as a cancer target capable of modulating cell survival following therapeutic treatment. Furthermore, it is obvious that dynamic flux in the state of phosphorylation of different sites of PGRMC1 is required for this vital or death-evading function. Therefore reagents capable of modulating the state of PGRMC1 phosphorylation will have a profound effect on cancer treatment, as well as the other indication areas of neural disorders, cardiovascular disease and inflammatory diseases where a role of PGRMC1 is obvious from the discussion above.

Conclusions:

PGRMC1 phosphorylation mutants affect growth and survival properties of cells, and are responsive to modulators of cholesterol abundance. It must be remembered that the effects induced by these mutants can be modulated inside in cells in a regulated manner by selective phosphorylation and sub-localisation of PGRMC1 in response to various stimuli. This study identifies PGRMC1 as a central regulator of cellular response to external challenges. Although we demonstrate these results using MCF7 breast cancer cells, the mechanisms of PGRMC1 protein interaction and phosphorylation are general as evidenced by the descriptions above, and PGRMC1 is therefore suitable for the diagnostic and therapeutic treatment of not only cancer but also nervous system disorders, diseases of inflammation, and cardiocascular disorders.

TABLE 1

| Experimental | | | | | Lymph | | | | Her2/ | |
|---|---|---|---|---|---|---|---|---|---|---|
| variable | | Tumor | RNA | Tumor | node | | ER | PR | neu- | Age of |
| designation | Pools | number | Quality | status | status | Grade | status | status | Status | patient |
| ER+ | ER + 1 | T378 | ok | 2 | 0 | 2 | 12 | 4 | 0 | 75 |
| | | T392 | ok | 2 | 0 | 2 | 12 | 2 | 2* | 61 |
| | ER + 2 | T460 | ok | 2 | 0 | 2 | 4 | 8 | 3 | 79 |
| | | T464 | ok | 2 | 0 | 2 | 12 | 8 | 0 | 50 |
| | ER + 3 | T288 | ok | 2 | 0 | 2 | 12 | 4 | 1 | 76 |
| | | T711 | ok | 4 | 2 | 3 | 8 | 6 | 0 | 65 |
| | ER + 4 | T712 | ok | 2 | 1 | 2 | 9 | 4 | 0 | 58 |
| | | T425 | ok | 2 | 1 | 2 | 12 | 0 | 0 | 78 |
| ER− | ER − 1 | T433 | ok | 2 | 0 | 2 | 0 | 0 | 1 | 42 |
| | | T443 | ok | 2 | 1 | 2 | 0 | 12 | 0 | 46 |
| | ER − 2 | T469 | ok | 1 | 0 | 2-3 | 0 | 0 | 3 | 50 |
| | | T470 | ok | 2 | 1 | 2 | 0 | 0 | 0 | 39 |
| | ER − 3 | T531 | ok | 2 | 0 | 2 | 0 | 0 | 0 | 58 |
| | | T558 | ok | 2 | 0 | 2-3 | 0 | 0 | 0 | 62 |
| | ER − 4 | T623 | ok | 1 | x | 2-3 | 0 | 0 | 1 | 42 |
| | | T640 | ok | 2 | 0 | 3 | 0 | 0 | 3 | 62 |

*no Gene Amplification detected
x: pNx regional lymphnodes cannot be assessed

TABLE 2

| Protein | Number | Experimental | | Genbank | PMF |
|---|---|---|---|---|---|
| Name | of Spots | PI | MW | AccNo | Score |
| Albumin | 19 spots | 5.8 | 73000 | gi\|23307793 | 87 |
| | | 5.3 | 71000 | gi\|6013427 | 54 |
| | | 5.3 | 67000 | | 72 |
| | | 5.3 | 66000 | | 58 |
| | | 5.4 | 83000 | | 75 |
| | | 5.4 | 72000 | | 93 |
| | | 5.4 | 72000 | | 96 |
| | | 5.4 | 62000 | | 85 |
| | | 5.4 | 60000 | | 70 |
| | | 5.5 | 72000 | | 112 |
| | | 5.5 | 71000 | | 158 |
| | | 5.5 | 70000 | | 86 |
| | | 5.6 | 71000 | | 136 |
| | | 5.6 | 71000 | | 144 |
| | | 5.6 | 50000 | | 63 |
| | | 5.7 | 104000 | | 94 |
| | | 5.7 | 71000 | | 88 |
| | | 5.7 | 71000 | | 90 |
| | | 5.7 | 71000 | | 128 |
| ATP synthase | 2 spots | 6.7 | 55000 | gi\|24660110 | 83 |
| | | 6.9 | 57000 | | 169 |
| Carbonic | 2 Spots | 6.5 | 27000 | gi\|1633065 | 78 |
| Ahydrase II | | 6.7 | 26000 | gi\|999651 | 74 |
| Cyclophilin A | 3 Spots | 6.8 | 16000 | gi\|1633054 | 87 |
| | | 7.4 | 15000 | | 81 |
| | | 7.0 | 16000 | | 85 |
| Fibrinogen | 5 spots | 6.1 | 59000 | gi\|399492 | 79 |
| beta | | 6.1 | 57000 | | 85 |
| | | 6.8 | 55000 | | 66 |
| | | 7.1 | 57000 | | 88 |
| | | 5.6 | 40000 | gi\|2781208 | 70 |
| HSP27 | 2 spots | 5.4 | 26000 | gi\|662841 | 123 |
| | | 5.5 | 26000 | | 125 |
| Keratin 7 | 3 spots | 5.2 | 55000 | gi\|30089956 | 97 |
| | | 5.2 | 54000 | | 112 |
| | | 5.3 | 56000 | | 273 |
| Keratin 8 | 4 spots | 5.3 | 56000 | gi\|39645331 | 154 |
| | | 5.1 | 49000 | gi\|4504919 | 102 |
| | | 5.2 | 49000 | | 215 |
| | | 5.4 | 55000 | | 419 |
| Keratin 9 | 4 spots | 5.1 | 67000 | gi\|435476 | 217 |
| | | 5.1 | 66000 | | 141 |
| | | 5.1 | 16000 | | 132 |
| | | 5.1 | 12000 | gi\|4557705 | 72 |
| Keratin 19 | 4 spots | 5.0 | 43000 | gi\|34783124 | 407 |
| | | 5.0 | 42000 | | 395 |
| | | 5.0 | 42000 | | 424 |
| | | 4.9 | 41000 | | 204 |
| Alpha 1 | 14 spots | 4.5 | 60000 | gi\|1942629 | 126 |
| antitrypsin | | 4.8 | 62000 | | 103 |
| | | 4.8 | 54000 | | 97 |
| | | 4.8 | 52000 | | 71 |
| | | 4.8 | 51000 | | 93 |
| | | 4.8 | 50000 | | 80 |
| | | 4.9 | 63000 | | 186 |
| | | 4.9 | 50000 | | 97 |
| | | 5.0 | 61000 | | 232 |
| | | 5.0 | 60000 | | 254 |
| | | 5.0 | 49000 | | 149 |
| | | 5.0 | 61000 | | 130 |
| | | 5.1 | 60000 | | 133 |
| | | 4.9 | 50000 | | 66 |
| Translation | 2 spots | 4.9 | 35000 | gi\|25453472 | 93 |
| elongation | | 5.0 | 35000 | | 70 |
| factor 1 delta | | | | | |
| mPR | 3 spots | 4.6 | 22000 | gi\|5729875 | 95 |
| | | 4.55 | 22000 | | 110 |
| | | 4.5 | 22000 | | 107 |
| Transferrin | 2 spots | 6.0 | 80000 | gi\|37747855 | 99 |
| receptor | | 6.2 | 81000 | gi\|4557871 | 73 |
| Transgelin | 2 spots | 7.8 | 22000 | gi\|4507359 | 95 |
| | | 8.5 | 21000 | | 137 |
| Vimentin | 3 spots | 4.8 | 47000 | gi\|4507895 | 336 |
| | | 5.0 | 56000 | | 531 |
| | | 4.9 | 45000 | | 150 |

### REFERENCES

[1] Jemal A, Siegel R, Ward E, Murray T, Xu J, Thun M J: Cancer statistics, 2007. CA Cancer J Clin 2007;57:43-66.
[2] Fisher B, Jeong J H, Dignam J, Anderson S, Mamounas E, Wickerham D L, Wolmark N: Findings from recent National Surgical Adjuvant Breast and Bowel Project adjuvant studies in stage I breast cancer. J Natl Cancer Inst Monogr 2001;62-66.
[3] Osborne C K: Steroid hormone receptors in breast cancer management. Breast Cancer Res Treat 1998;51:227-238.
[4] Henderson I C: Risk factors for breast cancer development. Cancer 15 Mar. 1993;71:2127-2140.
[5] Howell A, Osborne C K, Morris C, Wakeling A E: ICI 182,780 (Faslodex): development of a novel, “pure” antiestrogen. Cancer 15 Aug. 2000;89:817-825.
[6] McKeage K, Curran M P, Plosker G L: Fulvestrant: a review of its use in hormone receptor-positive metastatic breast cancer in postmenopausal women with disease progression following antiestrogen therapy. Drugs 2004;64:633-648.
[7] Platet N, Prevostel C, Derocq D, Joubert D, Rochefort H, Garcia M: Breast cancer cell invasiveness: correlation with protein kinase C activity and differential regulation by phorbol ester in estrogen receptor-positive and -negative cells. Int J Cancer 2 Mar. 1998;75:750-756.
[8] Thompson E W, Paik S, Brunner N, Sommers C L, Zugmaier G, Clarke R, Shima T B, Torri J, Donahue S, Lippman M E,: Association of increased basement membrane invasiveness with absence of estrogen receptor and expression of vimentin in human breast cancer cell lines. J Cell Physiol 1992;150:534-544.
[9] Fuqua S A, Cui Y: Estrogen and progesterone receptor isoforms: clinical significance in breast cancer. Breast Cancer Res Treat 2004;87 Suppl 1:S3-10.
[10] Price J E, Polyzos A, Zhang R D, Daniels L M: Tumorigenicity and metastasis of human breast carcinoma cell lines in nude mice. Cancer Res 1 Feb. 1990;50:717-721.
[11] Sorlie T, Perou C M, Tibshirani R, Aas T, Geisler S, Johnsen H, Hastie T, Eisen M B, van de R M, Jeffrey S S, Thorsen T, Quist H, Matese J C, Brown P O, Botstein D, Eystein L P, Borresen-Dale A L: Gene expression patterns of breast carcinomas distinguish tumor subclasses with clinical implications. Proc Natl Acad Sci U S A 11 Sep. 2001;98:10869-10874.
[12] Sorlie T, Tibshirani R, Parker J, Hastie T, Marron J S, Nobel A, Deng S, Johnsen H, Pesich R, Geisler S, Demeter J, Perou C M, Lonning P E, Brown P O, Borresen-Dale A L, Botstein D: Repeated observation of breast tumor subtypes in independent gene expression data sets. Proc Nati Acad Sci U S A 8 Jul. 2003;100:8418-8423.
[13] Abd El-Rehim D M, Pinder S E, Paish C E, Bell J, Blamey R W, Robertson J F, Nicholson R I, Ellis I O: Expression of luminat and basal cytokeratins in human breast carcinoma. J Pathol 2004;203:661-671.
[14] Nielsen T O, Hsu F D, Jensen K, Cheang M, Karaca G, Hu Z, Hernandez-Boussard T, Livasy C, Cowan D, Dressler L, Akslen L A, Ragaz J, Gown A M, Gilks C B, van de R M, Perou C M: Immunohistochemical and clinical characterization of the basal-like subtype of invasive breast carcinoma. Clin Cancer Res 15 Aug. 2004;10:5367-5374.
[15] Weigelt B, Glas A M, Wessels L F, Witteveen A T, Peterse J L, van't Veer L J: Gene expression profiles of primary breast tumors maintained in distant metastases. Proc Natl Acad Sci U S A 23 Dec. 2003;100:15901-15905.
[16] Dvorak H F: Tumors: wounds that do not heal. Similarities between tumor stroma generation and wound healing. N Engl J Med 25 Dec. 1986;315:1650-1659.
[17] Chang H Y, Sneddon J B, Alizadeh A A, Sood R, West R B, Montgomery K, Chi J T, van de R M, Botstein D, Brown P O: Gene expression signature of fibroblast serum response predicts human cancer progression: similarities between tumors and wounds. PLoS Biol 2004;2:E7.
[18] Cahill M A: Progesterone Receptor Membrane Component 1: an integrative review. J Ster Biochem Mol Biol 2007;104:In Press.
[19] Runko E, Kaprielian Z: Caenorhabditis elegans VEM-1, a novel membrane protein, regulates the guidance of ventral nerve cord-associated axons. J Neurosci 13 Oct. 2004;24:9015-9026.
[20] Anderson R G: The caveolae membrane system. Annu Rev Biochem 1998;67:199-225.
[21] Simons K, Ikonen E: Functional rafts in cell membranes. Nature 5 Jun. 1997;387:569-572.
[22] Sbaa E, Frerart F, Feron O: The double regulation of endothelial nitric oxide synthase by caveolae and caveolin: a paradox solved through the study of angiogenesis. Trends Cardiovasc Med 2005;15:157-162.
[23] Umebayashi K: The roles of ubiquitin and lipids in protein sorting along the endocytic pathway. Cell Struct Funct 2003;28:443-453.
[24] Sun Y: E3 ubiquitin ligases as cancer targets and biomarkers. Neoplasia 2006;8:645-654.
[25] Serafini T, Kennedy T E, Galko M J, Mirzayan C, Jessell T M, Tessier-Lavigne M: The netrins define a family of axon outgrowth-promoting proteins homologous to C. elegans UNC-6. Cell 12 Aug. 1994;78:409-424.
[26] de Castro F: Chemotropic molecules: guides for axonal pathfinding and cell migration during CNS development. News Physiol Sci 2003;18:130-136.
[27] Keino-Masu K, Masu M, Hinck L, Leonardo E D, Chan S S, Culotti J G, Tessier-Lavigne M: Deleted in Colorectal Cancer (DCC) encodes a netrin receptor. Cell 18 Oct. 1996;87:175-185.
[28] Shirasaki R, Mirzayan C, Tessier-Lavigne M, Murakami F: Guidance of circumferentially growing axons by netrin-dependent and -independent floor plate chemotropism in the vertebrate brain. Neuron 1996;17:1079-1088.
[29] Jia L, Emmons S W: Genes that control ray sensory neuron axon development in the Caenorhabditis elegans male. Genetics 2006;173:1241-1258.
[30] Kennedy T E: Cellular mechanisms of netrin function: long-range and short-range actions. Biochem Cell Biol 2000;78:569-575.
[31] Matsunaga E, Chedotal A: Repulsive guidance molecule/neogenin: a novel ligand-receptor system playing multiple roles in neural development. Dev Growth Differ 2004;46:481-486.
[32] Matsunaga E, Nakamura H, Chedotal A: Repulsive guidance molecule plays multiple roles in neuronal differentiation and axon guidance. J Neurosci 31 May 2006;26:6082-6088.
[33] Burgess R W, Jucius T J, Ackerman S L: Motor axon guidance of the mammalian trochlear and phrenic nerves: dependence on the netrin receptor Unc5c and modifier loci. J Neurosci 24 May 2006;26:5756-5766.
[34] Mehien P, Fume C: Netrin-1: when a neuronal guidance cue turns out to be a regulator of tumorigenesis. Cell Mol Life Sci 2005;62:2599-2616.
[35] Bredesen D E, Mehlen P, Rabizadeh S: Receptors that mediate cellular dependence. Cell Death Differ 2005;12:1031-1043.
[36] Arakawa H: Netrin-1 and its receptors in tumorigenesis. Nat Rev Cancer 2004;4:978-987.
[37] Xie Y, Hong Y, Ma X Y, Ren X R, Ackerman S, Mei L, Xiong W C: DCC-dependent phospholipase C signaling in netrin-1-induced neurite elongation. J Biol Chem 3 Feb. 2006;281:2605-2611.
[38] Culotti J G, Merz D C: DCC and netrins. Curr Opin Cell Biol 1998;10:609-613.
[39] Stein E, Zou Y, Poo M, Tessier-Lavigne M: Binding of DCC by netrin-1 to mediate axon guidance independent of adenosine A2B receptor activation. Science 9 Mar. 2001;291:1976-1982.
[40] Brose K, Tessier-Lavigne M: Slit proteins: key regulators of axon guidance, axonal branching, and cell migration. Curr Opin Neurobiol 2000;10:95-102.
[41] Zhu X J, Wang C Z, Dai P G, Xie Y, Song N N, Liu Y, Du Q S, Mei L, Ding Y Q, Xiong W C: Myosin X regulates netrin receptors and functions in axonal path-finding. Nat Cell Biol 2007;9:184-192.
[42] Gitai Z, Yu T W, Lundquist E A, Tessier-Lavigne M, Bargmann C I: The netrin receptor UNC-40/DCC stimulates axon attraction and outgrowth through enabled and, in parallel, Rac and UNC-115/AbLIM. Neuron 9 Jan. 2003;37:53-65.
[43] Forcet C, Stein E, Pays L, Corset V, Llambi F, Tessier-Lavigne M, Mehlen P: Netrin-1-mediated axon outgrowth requires deleted in colorectal cancer-dependent MAPK activation. Nature 23 May 2002;417:443-447.
[44] Xie Y, Ding Y Q, Hong Y, Feng Z, Navarre S, Xi C X, Zhu X J, Wang C L, Ackerman S L, Kozlowski D, Mei L, Xiong W C: Phosphatidylinositol transfer protein-alpha in netrin-1-induced PLC signalling and neurite outgrowth. Nat Cell Biol 2005;7:1124-1132.
[45] Barallobre M J, Pascual M, Del Rio J A, Soriano E: The Netrin family of guidance factors: emphasis on Netrin-1 signalling. Brain Res Brain Res Rev 2005;49:22-47.
[46] Bartoe J L, McKenna W L, Quan T K, Stafford B K, Moore J A, Xia J, Takamiya K, Huganir R L, Hinck L: Protein interacting with C-kinase 1/protein kinase Calpha-mediated endocytosis converts netrin-1-mediated repulsion to attraction. J Neurosci 22 Mar. 2006;26:3192-3205.
[47] Galko M J, Tessier-Lavigne M: Function of an axonal chemoattractant modulated by metalloprotease activity. Science 25 Aug. 2000;289:1365-1367.
[48] Pasquale E: Neurobiology. Turning attraction into repulsion. Science 25 Aug. 2000;289:1308-1310.
[49] Taniguchi Y, Kim S H, Sisodia S S: Presenilin-dependent “gamma-secretase” processing of deleted in colorectal cancer (DCC). J Biol Chem 15 Aug. 2003;278:30425-30428.
[50] Parent A T, Barnes N Y, Taniguchi Y, Thinakaran G, Sisodia S S: Presenilin attenuates receptor-mediated signaling and synaptic function. J Neurosci 9 Feb. 2005;25:1540-1549.
[51] Viero C, Mechaly I, Aptel H, Puech S, Valmier J, Bancel F, Dayanithi G: Rapid inhibition of Ca(2+) influx by neurosteroids in murine embryonic sensory neurones. Cell Calcium 10 Jun. 2006.
[52] Sakamoto H, Ukena K, Takemori H, Okamoto M, Kawata M, Tsutsui K: Expression and localization of 25-Dx, a membrane-associated putative progesterone-binding protein, in the developing Purkinje cell. Neuroscience 2004;126:325-334.
[53] Vetrivel K S, Cheng H, Kim S H, Chen Y, Barnes N Y, Parent A T, Sisodia S S, Thinakaran G: Spatial segregation of gamma-secretase and substrates in distinct membrane domains. J Biol Chem 8 Jul. 2005;280:25892-25900.
[54] Bouchard J F, Moore S W, Tritsch N X, Roux P P, Shekarabi M, Barker P A, Kennedy T E: Protein kinase A activation promotes plasma membrane insertion of DCC from an intracellular pool: A novel mechanism regulating commissural axon extension. J Neurosci 24 Mar. 2004;24:3040-3050.
[55] Fume C, Corset V, Herincs Z, Cahuzac N, Hueber A O, Mehlen P: The dependence receptor DCC requires lipid raft localization for cell death signaling. Proc Natl Acad Sci U S A 14 Mar. 2006;103:4128-4133.
[56] Nikolopoulos S N, Giancotti F G: Netrin-integrin signaling in epithelial morphogenesis, axon guidance and vascular patterning. Cell Cycle 2005;4:e131-e135.
[57] Cole S J, Bradford D, Cooper H M: Neogenin: A multi-functional receptor regulating diverse developmental processes. Int J Biochem Cell Biol 23 Nov. 2006.
[58] Park K W, Crouse D, Lee M, Karnik S K, Sorensen L K, Murphy K J, Kuo C J, Li D Y: The axonal attractant Netrin-1 is an angiogenic factor. Proc Natl Acad Sci U S A 16 Nov. 2004;101:16210-16215.
[59] Nguyen A, Cai H: Netrin-1 induces angiogenesis via a DCC-dependent ERK1/2-eNOS feed-forward mechanism. Proc Natl Acad Sci U S A 25 Apr. 2006;103:6530-6535.
[60] Hughes A L, Powell D W, Bard M, Eckstein J, Barbuch R, Link A J, Espenshade P J: Dap1/PGRMC1 binds and regulates cytochrome P450 enzymes. Cell Metab 2007;5:143-149.
[61] Brouet A, Sonveaux P, Dessy C, Moniotte S, Balligand J L, Feron O: Hsp90 and caveolin are key targets for the proangiogenic nitric oxide-mediated effects of statins. Circ Res 9 Nov. 2001;89:866-873.
[62] Kang J S, Yi M J, Zhang W, Feinleib J L, Cole F, Krauss R S: Netrins and neogenin promote myotube formation. J Cell Biol 8 Nov. 2004;167:493-504.
[63] Krauss R S, Cole F, Gaio U, Takaesu G, Zhang W, Kang J S: Close encounters: regulation of vertebrate skeletal myogenesis by cell-cell contact. J Cell Sci 1 Jun. 2005;118:2355-2362.
[64] Bazzoni G, Dejana E: Endothelial cell-to-cell junctions: molecular organization and role in vascular homeostasis. Physiol Rev 2004;84:869-901.
[65] Martin M, Simon-Assmann P, Kedinger M, Martin M, Mangeat P, Real F X, Fabre M: DCC regulates cell adhesion in human colon cancer derived HT-29 cells and associates with ezrin. Eur J Cell Biol 2006;85:769-783.
[66] Zhang A S, West A P, Jr., Wyman A E, Bjorkman P J, Enns C A: Interaction of hemojuvelin with neogenin results in iron accumulation in human embryonic kidney 293 cells. J Biol Chem 7 Oct. 2005;280:33885-33894.
[67] Valko M, Rhodes C J, Moncol J, Izakovic M, Mazur M: Free radicals, metals and antioxidants in oxidative stress-induced cancer. Chem Biol Interact 10 Mar. 2006;160:1-40.
[68] Slorach E M, Werb Z: Epithelial morphogenesis: Netrin comes to a sticky and terminal end. Curr Biol 17 Jun. 2003;13:R491-R493.
[69] Hinck L, Silberstein G B: Key stages in mammary gland development: the mammary end bud as a motile organ. Breast Cancer Res 2005;7:245-251.
[70] Strizzi L, Bianco C, Raafat A, Abdallah W, Chang C, Raafat D, Hirota M, Hamada S, Sun Y, Normanno N, Callahan R, Hinck L, Salomon D: Netrin-1 regulates invasion and migration of mouse mammary epithelial cells overexpressing Cripto-1 in vitro and in vivo. J Cell Sci 15 Oct. 2005;118:4633-4643.
[71] Huber M A, Kraut N, Beug H: Molecular requirements for epithelial-mesenchymal transition during tumor progression. Curr Opin Cell Biol 2005;17:548-558.
[72] Yang J, Mani S A, Donaher J L, Ramaswamy S, Itzykson R A, Come C, Savagner P, Gitelman I, Richardson A, Weinberg R A: Twist, a master regulator of morphogenesis, plays an essential role in tumor metastasis. Cell 25 Jun. 2004;117:927-939.
[73] Kang Y, Massague J: Epithelial-mesenchymal transitions: twist in development and metastasis. Cell 6 Aug. 2004;118:277-279.
[74] Vernon A E, LaBonne C: Tumor metastasis: a new twist on epithelial-mesenchymal transitions. Curr Biol 7 Sep. 2004;14:R719-R721.
[75] van', V, Dai H, van d, V, He Y D, Hart A A, Mao M, Peterse H L, van der K K, Marton M J, Witteveen A T, Schreiber G J, Kerkhoven R M, Roberts C, Linsley P S, Bernards R, Friend S H: Gene expression profiling predicts clinical outcome of breast cancer. Nature 31 Jan. 2002;415:530-536.
[76] Lee J E, Kim H J, Bae J Y, Kim S W, Park J S, Shin H J, Han W, Kim S W, Kang K S, Noh D Y: Neogenin expression may be inversely correlated to the tumorigenicity of human breast cancer. BMC Cancer 2005;5:154.
[77] Velardo M J, Burger C, Williams P R, Baker H V, Lopez M C, Mareci T H, White T E, Muzyczka N, Reier P J: Patterns of gene expression reveal a temporally orchestrated wound healing response in the injured spinal cord. J Neurosci 29-9-2004;24:8562-8576.
[78] Heaton J H, Dlakic W M, Dlakic M, Gelehrter T D: Identification and cDNA cloning of a novel RNA-binding protein that interacts with the cyclic nucleotide-responsive sequence in the Type-1 plasminogen activator inhibitor mRNA. J Biol Chem 2 Feb. 2001;276:3341-3347.
[79] Song H J, Poo M M: Signal transduction underlying growth cone guidance by diffusible factors. Curr Opin Neurobiol 1999;9:355-363.
[80] Corset V, Nguyen-Ba-Charvet K T, Forcet C, Moyse E, Chedotal A, Mehlen P: Netrin-1-mediated axon outgrowth and cAMP production requires interaction with adenosine A2b receptor. Nature 12 Oct. 2000;407:747-750.
[81] Ming G, Henley J, Tessier-Lavigne M, Song H, Poo M: Electrical activity modulates growth cone guidance by diffusible factors. Neuron 2001;29:441-452.
[82] Shewan D, Dwivedy A, Anderson R, Holt C E: Age-related changes underlie switch in netrin-1 responsiveness as growth cones advance along visual pathway. Nat Neurosci 2002;5:955-962.
[83] Nishiyama M, Hoshino A, Tsai L, Henley J R, Goshima Y, Tessier-Lavigne M, Poo M M, Hong K: Cyclic AMP/GMP-dependent modulation of Ca2+ channels sets the polarity of nerve growth-cone turning. Nature 26 Jun. 2003;423:990-995.
[84] Hand R A, Craven R J: Hpr6.6 protein mediates cell death from oxidative damage in MCF-7 human breast cancer cells. J Cell Biochem 15 Oct. 2003;90:534-547.
[85] Swanson K M, Hohl R J: Anti-cancer therapy: targeting the mevalonate pathway. Curr Cancer Drug Targets 2006;6: 15-37.
[86] Crudden G, Chitti R E, Craven R J: Hpr6 (heme-1 domain protein) regulates the susceptibility of cancer cells to chemotherapeutic drugs. J Pharmacol Exp Ther 2006;316:448-455.
[87] Dimitroulakos J, Nohynek D, Backway K L, Hedley D W, Yeger H, Freedman M H, Minden M D, Penn L Z: Increased sensitivity of acute myeloid leukemias to lovastatin-induced apoptosis: A potential therapeutic approach. Blood 15 Feb. 1999;93:1308-1318.

1. Use of at least one isoform of progesterone receptor membrane component 1 (PGRMC1 ) as diagnostic marker and/or therapeutic target for diseases associated with neogenin and/or DCC (Deleted in colorectal cancer).

2. Use of at least one isoform of progesterone receptor membrane component 1 (PGRMC 1 ) for the manufacture of a medicament for treatment of diseases associated with neogenin and/or DCC.

3. Use of at least one reagent that influences the function of at least one isoform of progesterone receptor membrane component 1 (PGRMC 1) for the manufacture of a medicament for treatment of diseases associated with neogenin and/or DCC.

4. Use according to claim 1, characterized in that at least two or three isoforms are used as diagnostic marker and/or therapeutic target or in the manufacture of the medicament.

5. Use according to claim 1, characterized in that said diseases comprise neuronal disorders, preferably memory disorders, in particular in mammalians.

6. Use according to claim 1, characterized in that said diseases comprise cancer of the nervous system.

7. Use according to claim 1, characterized in that said diseases comprise cancer, in particular breast cancer.

8. Use according to claim 1, characterized in that said diseases comprise inflammatory diseases, in particular atherosclerosis or rheumatoid arthritis.

9. Use according to claim 1, characterized in that said diseases comprise cardiovascular diseases.

10. Use according to claim 6, characterized in that at least one of said isoforms of PGRMC 1 has a significantly increased abundance in cancer cells of said cancers.

11. Use according to claim 1, characterized in that said isoforms of PGRMC 1 differ from each other in their phosphorylation status.

12. Use according to claim 10, characterized in that said cancer cells are negative for the estrogen receptor (ER−).

13. Use according to claim 10, characterized in that said cancer cells are negative for the progesterone receptor (PR−).

14. Use of at least one isoform of progesterone receptor membrane component 1 (PGRMC1) for influencing, in particular increasing, abundance or subcellular localisation of at least one class of molecules interacting with PGRMC1.

15. Use of at least one isoform of progesterone receptor membrane component 1 (PGRMC1) for influencing, in particular decreasing, abundance or subcellular localisation of at least one class of molecules interacting with PGRMC1.

16. Use according to claim 14, characterized in that said class of molecule is at least one of Deleted in colorectal cancer (DCC) or Neogenin.

17. Use according to claim 14, characterized in that said class of molecule is a ubiquitin ligase.

18. Use according to claim 14, characterized in that said class of molecule is a polyubiquitinase.

19. Use according to claim 3, characterized in that said at least one reagent increases or decreases the function or changes the subcellular locality of of PGRMC1.

20. Use according to claim 3, characterized in that said at least one reagent is a reagent for influencing, in particular increasing or decreasing, the phosphorylation status of the at least one isoform of PGRMd.

21. Use according to claim 3, characterized in that said reagent is an antibody or small molecule affinity reagent directed against PGRMC1.

22. Assay kit for diagnosis and/or therapy of diseases associated with neogenin and/or DCC, comprising at least one isoform of progesterone receptor membrane component 1 (PGRMC1).

23. Assay kit for diagnosis and/or therapy of diseases associated with neogenin and/or DCC, comprising at least one reagent that influences the function of at least one isoform of progesterone receptor membrane component 1 (PGRMC1).

24. Use of at least one isoform of progesterone receptor membrane component 1 (PGRMC1) as diagnostic marker in diagnosis for diseases associated with aberrant biological pheno-types, wherein the phosphorylation status of the at least one isoform of progesterone receptor membrane component 1 (PGRMC1) is determined/and or estimated.

25. Use according to claim 24, characterized in that the phosphorylation status is determined and/or estimated by analysis of proteins that are involved, in particular differentially involved, in protein interaction or multi-protein complexes with either phosphorylated or non-phosphorylated at least one isoform of progesterone receptor membrane component 1 (PGRMC1).

26. Use of at least one reagent for influencing, in particular increasing or inhibiting, the abundance and/or activity of isoforms of proteins for diagnosis and/or therapy of diseases associated with aberrant biological phenotypes, wherein said proteins are involved, in particular differentially involved, in protein interaction or multi-protein complexes with either at least one isoform of phosphorylated or non-phosphorylated progesterone receptor membrane component 1 (PGRMC1)

27. Use according to claim 26, characterized in that said diseases, in particular subgroups thereof, comprise cancer, especially breast cancer or prostate cancer, neurodegenerative diseases, infertility, inflammatory, immunological, respiratory, pulmonary diseases, and/or diseases associated with the rate of biological aging or with beneficial or detrimental alterations of the level of the process of autophagy.

28. A method of treating a disease associated with neogenin and/or DCC (Deleted in colorectal cancer) in a patient in need of such treatment, comprising administering to said patient an effective amount of at least one isoform of progesterone receptor membrane component 1 (PGRMC1).
